(19) United States
(12) Patent Application Publication
(10) Pub. No.: US 2025/0281488 A1
(43) Pub. Date: Sep. 11, 2025

(54) VCP INHIBITORS AND USES THEREOF FOR TREATMENT

(71) Applicant: The Francis Crick Institute Limited, London (GB)

(72) Inventors: Rickie PATANI, London (GB); Jasmine HARLEY, Rugeley Straffordshire (GB)

(21) Appl. No.: 18/576,830

(22) PCT Filed: Jul. 7, 2022
(86) PCT No.: PCT/EP2022/069011

§ 371 Date: Jan. 5, 2024

(30) Foreign Application Priority Data
Jul. 7, 2021 (GB) 2109830.6

(51) Int. Cl. A61K 31/517; A61K 31/121; A61K 31/4178; A61K 31/428; A61K 31/4439; A61K 31/496; A61K 31/519; A61K 31/538; A61P 25/28

(52) U.S. Cl. CPC A61K 31/517; A61K 31/121; A61K 31/4178; A61K 31/428; A61K 31/4439; A61K 31/496; A61K 31/519; A61K 31/538; A61P 25/28

(57) ABSTRACT

The present invention relates to inhibitors of Valosin-containing protein (VCP or p97) and the use thereof in the treatment or prevention of diseases such as amyotrophic lateral sclerosis (ALS). In particular the present invention provides VCP inhibitors for use in a method of treating or preventing ALS wherein the subject has been identified as not having a disease-causing genetic mutation in a VCP gene (non-VCP-associated ALS). The invention also relates to methods of identifying a patient as not having a disease-causing mutation in a VCP gene.

### CROSS-REFERENCE TO RELATED APPLICATIONS

This application is a national phase application filed under 35 U.S.C. § 371 claiming benefit to International Patent Application No. PCT/EP2022/069011, filed Jul. 7, 2022, which claims the benefit of priority from United Kingdom Patent Application No. 2109830.6, filed Jul. 7, 2021, the contents of each of which is hereby incorporated by reference in its entirety.

### REFERENCE TO A “SEQUENCE LISTING” SUBMITTED AS AN XML FILE

The Sequence Listing written in the xml file titled: “206189-0050-00US_SequenceListingv2.xml”; created on Aug. 12, 2024, and 2,733 bytes in size, is hereby incorporated by reference.

### TECHNICAL FIELD

The present invention relates to inhibitors of Valosin-containing protein (VCP or p97) and the use thereof in the treatment or prevention of diseases such as amyotrophic lateral sclerosis (ALS). In particular the present invention provides VCP inhibitors for use in a method of treating or preventing ALS wherein the subject has been identified as not having a disease-causing genetic mutation in a VCP gene (non-VCP-associated ALS). The invention also relates to methods of identifying a patient as not having a disease-causing mutation in a VCP gene.

### BACKGROUND

Amyotrophic lateral sclerosis (ALS) is an invariably fatal neurological disease in which there is selective and progressive degeneration of motor neurons. Deregulated RNA metabolism, and in particular RNA binding protein (RBP) subcellular localization and function, play a pivotal role in ALS pathogenesis. RBPs orchestrate the RNA life cycle, regulating transcription, splicing, RNA localisation, function and decay.

Some ALS-causing gene mutations encode RBPs, including transactive response DNA-binding protein 43 (TARDBP, which encodes TDP-43), fused in sarcoma/translocated in liposarcoma (FUS/TLS or FUS) and heterogeneous nuclear ribonucleoprotein A1 (hnRNPA1). Subcellular mislocalization of RBPs is also a pathological hallmark of ALS, with TDP-43 mislocalized from the nucleus to the cytoplasm in 97% of ALS cases. [1]

More recently, widespread SFPQ and FUS mislocalization across different ALS models and sporadic ALS post-mortem tissue has also been reported [2,3]. Accumulation of RBPs in the cytoplasm likely contributes to the formation of RBP oligomers and fibrillar pathological cytoplasmic inclusions seen in ALS [4,5]. As one RBP alone can bind to many thousands of RNA targets, a disturbance in even a single RBP potentially has a broad and diverse impact on RNA metabolism [6].

Valosin-containing protein (VCP or p97) is an abundant AAA+ ATPase (ATPases associated with diverse cellular activities) with a large variety of intracellular functions, that encompass almost all aspects of cellular physiology. VCP functions include protein homeostasis, mitochondrial quality control, and apoptosis [7]. The structure of VCP is important to its many functions; it is a hexameric protein and each subunit has an N-terminal domain, two ATPase domains (D1 and D2) and a disordered C-terminal domain. Autosomal-dominant VCP mutations account for about 2% of familial ALS cases [8].

Although limited, pathogenic variants in VCP have also been identified in sporadic cases of the disease [9]. Due to its role in many cellular pathways, disruption to VCP function can lead to several forms of disease. For example, mutations in VCP have also been identified in other neurodegenerative diseases including, Inclusion Body Myopathy, Paget's disease and Frontotemporal Dementia (IBMPFD).

Pathogenic mutations of VCP are most commonly found in the N terminal domain, which is responsible for cofactor and ubiquitinated substrate binding, but are also present in the D1 and D2 domains [10]. The majority of VCP mutations have biochemically been shown to associate with a normal or increased ATPase activity in cellular models, with the R155C mutation shown to have more than double the activity of its wildtype counterpart [11]. However, it remains controversial whether disease mutants with increased ATPase activity cause disease through dominant active or dominant negative mechanisms. Additionally, this has yet to be systematically addressed in patient-derived motor neurons, which possess the advantage of conveying mutations at pathophysiological levels.

Missense mutations of VCP account for 1-2% of familial ALS, but can additionally cause an autosomal dominant disease known as inclusion body myopathy, Paget's disease and frontotemporal dementia (IBMPFD). ALS-causing VCP mutations recapitulate key hallmarks of sporadic ALS including nuclear-to-cytoplasmic mislocalization of key RBPs including TDP-43, FUS and SFPQ. [2,3,8,29]. However, the mechanism by which VCP mutations lead to RBP mislocalization in ALS has remained elusive.

It has previously been reported that human induced pluripotent stem cells (iPSCs) can be robustly differentiated into highly enriched and functionally validated spinal cord motor neurons in which time-resolved molecular phenotypes of VCP-related ALS have been identified [2,3,12,13]. Here, we used this established human stem cell model of VCP-mutation related ALS to first examine the nucleocytoplasmic distribution of key RBPs compared to control motor neurons.

We reveal that TDP-43, FUS and SFPQ exhibit aberrant reduced nuclear to cytoplasmic ratios in the VCP mutant motor neurons, which extends to an aberrant presence within the neurites. We found that treatment of control motor neurons with a targeted VCP D2 ATPase inhibitor does not recapitulate ALS RBP mislocalization phenotypes, arguing against a loss of its function in disease. Importantly, we find that in VCP mutant motor neurons the nuclear-to-cytoplasmic mislocalization of both TDP-43 and FUS and the nuclear-to-neurite mislocalization of TDP-43, FUS and SFPQ is reversible by treatment with a pharmacological inhibitor of the VCP D2 ATPase domain.

Cumulatively these findings support a model whereby VCP mutations cause an increase in D2 ATPase activity, which in turn leads to mislocalization of TDP-43, FUS and SFPQ from the nucleus to the cytosol. Our study raises the prospect of harnessing FDA approved VCP inhibitors that target the D2 ATPase domain in the treatment of VCP-related ALS.

### SUMMARY OF THE INVENTION

This summary introduces concepts that are described further in the detailed description. It should not be used to identify essential features of the claimed subject matter, nor to limit the scope of the claimed subject matter.

As discussed in Example 3, the present invention is the first disclosure that pharmacological inhibition of the VCP D2 ATPase domain does not induce ALS phenotypes in healthy human motor neurons (e.g. motor neurons not having a disease-causing mutation in a VCP gene). Rather, the present invention is the first demonstration that a VCP inhibitor can reverse the mislocalization of RNA binding protein in control (non-VCP-mutant) motor neurons. Accordingly, the present application provides the first disclosure that a VCP inhibitor could be used treating or preventing ALS in a subject that has been identified as not having a disease-causing mutation in a VCP gene (non-VCP-associated ALS).

A critical result is the change in localisation of TDP-43 and FUS shown in Example 3. Mislocalization of TDP-43 is a key disease hallmark of ALS. It is mislocalized from nucleus to the cytoplasm in over 97% of ALS cases. FIG. 2 (as discussed in Example 3) demonstrated for the first time that a VCP inhibitor can enhance nuclear localization of TDP-43 in healthy human motor neurons and is evidence of a treatment pathway for non-VCP-associated ALS.

The present invention provides a VCP (Valosin-containing protein) inhibitor for use in a method of treating or preventing amyotrophic lateral sclerosis (ALS) in a subject. In some embodiments, the ALS is non-VCP-associated ALS.

In some embodiments, the subject has not been identified as having a disease-causing mutation in a VCP gene. In some embodiments, the subject has been identified as not having a disease-causing mutation in a VCP gene. In some embodiments, the ALS is non-VCP-associated ALS.

In some embodiments, the subject might not have certain disease-causing genetic mutations in a VCP gene. In some embodiments, the subject has been identified as not having a disease-causing genetic mutation in a VCP gene at any of positions R155 and R191. In some embodiments, the subject has been identified as not having a disease-causing genetic mutation in a VCP gene selected from the list consisting of: R155C and R191Q. In some embodiments, the subject has been identified as not having a disease-causing genetic mutation in a VCP gene at any of positions R95, I114, I151, R155, G156, M158, R159, R191, N387, N401, R487, D592, R662 and N750. In some embodiments, the subject has been identified as not having any disease-causing genetic mutation in a VCP gene selected from the list consisting of: R95C, R95G, I114V, I151V, R155H, R155C, G156C, M158V, R159G, R159C, R159H, R191G, R191Q, N387T, N401S, R487H, D592N, R662C and N750S.

In some embodiment, the subject may have one or more disease-causing genetic mutations in a TARDBP gene. In some embodiments, the subject has been identified as having one or more disease-causing genetic mutations in a TARDBP gene.

In some embodiments, the amyotrophic lateral sclerosis is associated with reduction in the nuclear-to-cytoplasmic ratios of one or more of TDP-43, FUS and/or SFPQ. In some embodiments, the VCP inhibitor ameliorates one or more symptoms associated with reduction in the nuclear-to-cytoplasmic ratios of one or more of TDP-43, FUS and/or SFPQ.

In some embodiments, the amyotrophic lateral sclerosis is associated with reduction in the nuclear-to-cytoplasmic ratio of TDP-43. In some embodiments, the VCP inhibitor ameliorates one or more symptoms associated with reduction in the nuclear-to-cytoplasmic ratios of TDP-43.

In some embodiments, the amyotrophic lateral sclerosis is associated with reduction in the nuclear-to-cytoplasmic ratio of FUS. In some embodiments, the VCP inhibitor ameliorates one or more symptoms associated with reduction in the nuclear-to-cytoplasmic ratios of FUS.

In some embodiments, the amyotrophic lateral sclerosis is associated with reduction in the nuclear-to-cytoplasmic ratio of SFPQ. In some embodiments, the VCP inhibitor ameliorates one or more symptoms associated with reduction in the nuclear-to-cytoplasmic ratios of SFPQ.

In some embodiments, treating or preventing ALS comprises partial or complete alleviation, amelioration, relief, inhibition, delaying onset, reducing severity and/or incidence of neurological impairment in a patient suffering from or susceptible to ALS. In some embodiments, neurological impairment comprises symptoms associated with impairment of the central nervous system such as one or more of developmental delay, progressive cognitive impairment, hearing loss, impaired speech development, deficits in motor skills, hyperactivity, aggressiveness and/or sleep disturbances.

In some embodiments, treating or preventing ALS with a VCP inhibitor results in an improvement or amelioration of one or more neurological impairment symptoms by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%, as compared to the neurological impairment symptoms in the absence of a VCP inhibitor. In some embodiments, the treating or preventing ALS with a VCP inhibitor results in an improvement or amelioration of one or more neurological impairment symptoms by more than about 50%, about 80%, about 90% or about 95%.

In some embodiments, the VCP inhibitor inhibits the D2 ATPase domain of VCP.

In some embodiments, the VCP inhibitor is selected from the group consisting of: ML240 (2-(2-Amino-1H-benzimidazole-1-yl)-8-methoxy-N-(phenylmethyl)-4-quinazolinamine), ML241, 2-anilino-4-aryl-1,3-thiazoles, 3,4-methylenedioxy-6-nitrostyrene, DBeQ (N2,N4-dibenzylquinazo-line-2,4-diamine), CB-5083 (1-[7,8-dihydro-4-[(phenylmethyl)amino]-5H-pyrano[4,3-d]pyrimidin-2-yl]-2-methyl-1H-indole-4-carboxamide), CB-5339 (1-[4-(Benzylamino)-5,6,7,8-tetrahydropyrido[2,3-d]pyrimidin-2-yl]-2-methylindole-4-carboxamide), UPCDC-30245 (1-(3-(5-Fluoro-1H-indol-2-yl)phenyl)-N-(2-(4-isopropylpiperazin-1-yl)ethyl)piperidin-4-amine), NMS-873, NMS-859, Eeyarestatin I and Xanthohumol.

In some embodiments, the VCP inhibitor is ML240 (2-(2-Amino-1H-benzimidazole-1-yl)-8-methoxy-N-(phenylmethyl)-4-quinazolinamine).

In some embodiments, the VCP inhibitor is CB-5083 (1-[7,8-dihydro-4-[(phenylmethyl)amino]-5H-pyrano[4,3-d]pyrimidin-2-yl]-2-methyl-1H-indole-4-carboxamide) or CB-5339 (1-[4-(Benzylamino)-5,6,7,8-tetrahydropyrido[2,3-d]pyrimidin-2-yl]-2-methylindole-4-carboxamide).

The present invention also provides a method of diagnosing a subject as having or being suspected of having non-VCP-associated ALS comprising determining whether the subject has a disease-causing mutation in a VCP gene and providing a diagnosis of non-VCP-associated ALS based on the absence of a disease-causing mutation in a VCP gene.

In some embodiments, the method comprises identifying an absence of a disease-causing genetic mutation in a VCP gene at any of positions R95, I114, I151, R155, G156, M158, R159, R191, N387, N401, R487, D592, R662 and N750. In some embodiments, the method comprises identifying an absence of any disease-causing genetic mutation in a VCP gene selected from the list consisting of: R95C, R95G, I114V, I151V, R155H, R155C, G156C, M158V, R159G, R159C, R159H, R191G, R191Q, N387T, N401S, R487H, D592N, R662C and N750S.

The present invention also provides a VCP inhibitor for use in a method of treating or preventing non-VCP-associated ALS in a subject, comprising diagnosing a patient as having or as being suspected of having non-VCP-associated ALS using a method according to a method of the invention, and administering a VCP inhibitor to the patient.

The present invention also provides a VCP inhibitor for use in a method of treating or preventing non-VCP-associated ALS in a subject, wherein the patient has been determined as having or as being suspected of having non-VCP-associated ALS using a method according to a method of the invention, and administering a VCP inhibitor to the patient.

The present invention also provides a pharmaceutical composition comprising a VCP inhibitor for use in a method of treating or preventing amyotrophic lateral sclerosis (ALS), optionally wherein the pharmaceutical composition comprises one or more excipients.

The present invention also provides a method of treating or preventing amyotrophic lateral sclerosis (ALS) comprising administering a VCP inhibitor to a subject in need thereof. In some embodiments, the subject has not been identified as having a disease-causing mutation in a VCP gene. In some embodiments, the subject has been identified as not having a disease-causing mutation in a VCP gene. In some embodiments, the ALS is non-VCP-associated ALS. The VCP inhibitor may be administered in a therapeutically effective amount.

The present invention also provides a kit for diagnosing a subject as having or being suspected of having non-VCP-associated ALS comprising a means of determining whether the subject has a disease-causing mutation in a VCP gene. In some embodiments, the kit further comprises one or more containers containing one or more VCP inhibitors and, optionally informational material. In some embodiments, the informational material comprises directions for use of the kit in the diagnosis and/or treatment of non-VCP-associated ALS.

### BRIEF DESCRIPTION OF THE DRAWINGS

Embodiments of the present invention will now be described, by way of example only, with reference to the accompanying drawings, in which:

FIG. 1—TDP-43 and SFPQ mislocalization in VCP mutant motor neurons. A) TDP-43 immunolabeling in control and VCP mutant motor neurons. B) Individual cell analysis of TDP-43 nuclear:cytoplasmic ratio identifies VCP mutant motor neurons display a loss in the nuclear:cytoplasmic ratio (N/C). C) TDP-43 quantification in the neurites of motor neurons show VCP mutant motor neurons have a loss in the nuclear:neurite ratio (Nu/Ne). D) SFPQ immunolabeling in control and VCP mutant motor neurons. E) Individual cell analysis of SFPQ nuclear:cytoplasmic ratio shows there is a small but significant loss in VCP mutant motor neurons. F) SFPQ quantification in the neurites of motor neurons identifies that VCP mutant motor neurons have a decrease in the nuclear:neurite ratio. G) Immunolabeling of hnRNPA1 in control and VCP mutant motor neurons. H) Individual cell quantification of hnRNPA1 shows there is no difference in the nuclear:cytoplasmic ratio in control and VCP mutant motor neurons. I) hnRNPK localisation in control and VCP mutant motor neurons. J) Quantification of hnRNPK shows no difference in the nuclear:cytoplasmic ratio in control and VCP mutant motor neurons. Scale bar=10 μm. Data is collected from 3 control cell lines and 4 VCP mutant lines. For graphs B, E, H and J data is shown as a violin plot, with each data point representing a well from 6 independent experimental repeats (CTRL n=34, VCP n=45) with the p value shown from an unpaired T test. Approximately the following number of cells were analysed; B) CTRL1:10,000, CTRL2:10,000, CTRL3:14,000, MUT1:13,000, MUT2:15,000, MUT3:14,000, MUT4:11,000, E) CTRL1:9000, CTRL2:10,000, CTRL3:13,000, MUT1:12,000, MUT2:14,000, MUT3:14,000, MUT4:12,000, H) CTRL1:9000, CTRL2:10,000, CTRL3:13,000, MUT1:12,000, MUT2:12,000, MUT3:13,000, MUT4:9000, J) CTRL1:9000, CTRL2:9000, CTRL3:12,000, MUT1:11,000, MUT2:12,000, MUT3:12,000, MUT4:9000. For graphs C and F data is collected from 3 independent experiments from 3 control and 4 VCP mutant lines, with >5000 neurons analysed for each cell line. Data is shown as a violin plot with data points representing fields of view and the p value calculated from a Mann-Whitney test. All data is normalised to the average of the control values in each experimental repeat.

FIG. 2—Pharmacological inhibition of VCP D2 ATPase does not recapitulate ALS RBP mislocalization phenotypes in control motor neurons. A) Control motor neurons treated with 1 μM of ML240 (2-(2-Amino-1H-benzimidazole-1-yl)-8-methoxy-N-(phenylmethyl)-4-quinazolinamine) immunolabeled with TDP-43 and βIII-tubulin and a DAPI stain. B) Individual cell quantification of TDP-43 displayed control motor neurons treated with ML240 results in an increase in the nuclear:cytoplasmic ratio (N/C). C) There was no difference in the nuclear:neurite ratio (Nu/Ne) of TDP-43 upon ML240 treatment. D) Control motor neurons treated with 1 μM of ML240 immunolabeled with FUS and βIII-tubulin and a DAPI stain. E) Treatment of ML240 to control motor neurons showed no difference in the nuclear:cytoplasmic localisation of FUS. F) A small increase in the nuclear:neurite ratio of FUS upon ML240 treatment was observed. G) There was no difference in the nuclear:cytoplasmic ratio or H) nuclear:neurite ratio of SFPQ upon ML240 treatment. I) There was no difference in the nuclear:cytoplasmic ratio of hnRNPA1 or J) hnRNPK upon ML240 treatment in control motor neurons. Scale bar=10 μm. Data is shown as violin plots normalised to control untreated values in each experimental repeat. Data is collected from 3 control lines across 3 independent experimental repeats using approximately the following number of cells in both untreated and treated conditions; CTRL1:3000, CTRL2:6000, CTRL3:6000. For graphs B, E, G, I, J; each data point represents a well (UT n=16, ML240 n=16) and the p value is calculated from an unpaired t-test, for graphs C, F, H; each data point represents a field of view and the p value is calculated from a Mann-Whitney test.

FIG. 3—Inhibition of VCP D2-ATPase domain reverses TDP-43, FUS and SFPQ mislocalization phenotypes in VCP mutant motor neurons. A) VCP mutant motor neurons treated with 1 μM of ML240 immunolabeled with TDP-43 and βIII-tubulin. B) Cell by cell quantification of the nuclear:cytoplasmic ratio (N/C) shows VCP motor neurons have a loss in the nuclear:cytoplasmic ratio that is increased above control values upon ML240 treatment. C) Quantification of TDP-43 in the neurites shows an increased nuclear:neurite ratio (Nu/Ne) upon ML240 treatment. D) FUS and R111-tubulin immunolabeling in VCP mutant motor neurons treated with ML240. E) Quantification of FUS in the nucleus and cytoplasm identify an increase in the nuclear:cytoplasmic ratio upon ML240 treatment in VCP mutant motor neurons. F) Quantification of FUS in the neurites shows an increase of the nuclear:neurite ratio to control values upon ML240 treatment. G) VCP mutant motor neurons treated with ML240 and immunolabeled with SFPQ and R111-tubulin. H) Treatment of ML240 results in no change in the subcellular distribution of SFPQ when examining the nuclear:cytoplasmic ratio I) but an increase when examining the nuclear:neurite ratio. J) Quantification of hnRNPA1 shows no change in the nuclear:cytoplasmic ratio upon ML240 treatment in VCP mutant motor neurons. K) Quantification of hnRNPK shows no change in the nuclear:cytoplasmic ratio upon ML240 treatment in VCP mutant motor neurons. Scale bars=10 μm. Data is collected from 3 independent experimental repeats from 4 VCP ALS-mutant lines analysing approximately the following number of cells; MUT1:7000, MUT2:6000, MUT3:7000, MUT4:6000. Data is normalised to control untreated values for each experimental repeat. Data is shown as violin plots with each data point representing a well in graphs B, E, H, J and K (UT n=24, ML240 n=24) and a field in graphs C, F and I. Graphs B, E, H and K; p value is calculated from an unpaired t-test, for graphs C, F, I and J; p value is calculated from a Mann-Whitney test.

FIG. 4—Graphical depiction of the localisation of TDP-43, FUS and SFPQ in control motor neurons and mutated neurons and the effect of VCP D2 ATPase inhibition.

FIG. 5—Example images of the neuronal segmentation used in the image analysis. A) Examples of nuclear and cytoplasmic compartments used in the nuclear:cytoplasmic ratio analysis. The nuclear:cytoplasmic ratio is calculated per cell. B) Example of the nuclear and neurite compartments used in the nuclear:neurite ratio analysis. The nuclear:neurite ratio is calculated per field of view.

FIG. 6—Motor neuron characterisation. Representative images of control and VCP mutant iPSC-derived motor neurons, immunolabeled with motor neuron specific markers SMI-32 and ChAT and neuronal marker βIII-tubulin. Scale bar=20 μm.

FIG. 7—Compartmental analysis of TDP-43 and FUS in VCP mutant motor neurons. A) Nuclear compartmental analysis shows VCP mutant motor neurons have a loss of TDP-43 in the nucleus when compared to DAPI. B) Neurite compartmental analysis shows VCP mutant motor neurons have a gain of TDP-43 in the neuronal process when compared to neuronal marker βIII-tubulin. C) Compartmental analysis shows a loss of SFPQ protein in the nucleus in VCP mutant motor neurons. D) Compartmental analysis shows a gain of SFPQ in the neurites of VCP mutant motor neurons. Data is shown as violin plots normalised to control untreated values in each experimental repeat. Data is collected from 3 control lines from 6 wells across 3 independent experimental repeats. Data is plotted per field of view and the p value is calculated from a Mann-Whitney test.

FIG. 8—Western blot analysis shows TDP-43, FUS and SFPQ protein levels do not change upon inhibition of VCP D2 ATPase domain. A) Representative immunoblot of SPFQ, FUS and TDP-43 in control and VCP mutant MNs untreated and treated with ML240 1 μM. B) Quantification of SPFQ, FUS and TDP-43 from 3 control and 3 VCP mutant lines normalised to GAPDH showed ML240 treatment does not change overall protein levels.

FIG. 9—Details of the iPSC cell lines used in the study. MUT1 and MUT2 are cell lines having a R191Q mutation in VCP; MUT3 and MUT4 are cell lines having a R155C mutation in VCP; and MUT5 and MUT6 are cell lines having a G298S mutation in TARDBP.

FIG. 10—Exemplary VCP protein sequence. This figure discloses an exemplary human VCP protein sequence (UniProtKB—P55072).

FIG. 11—Compartmental analysis of TDP-43 in VCP-mutant and TARDBP-mutant motor neurons. A) Individual cell quantification of TDP-43 showed control motor neurons treated with DBEQ results in an increase in the nuclear:cytoplasmic ratio (N/C). B) Individual cell quantification of TDP-43 showed control motor neurons treated with CB-5083 results in an increase in the nuclear:cytoplasmic ratio (N/C). C) Individual cell quantification of TDP-43 showed VCP mutant motor neurons treated with DBEQ results in an increase in the nuclear:cytoplasmic ratio (N/C). D) Individual cell quantification of TDP-43 showed VCP mutant motor neurons treated with CB5083 results in an increase in the nuclear:cytoplasmic ratio (N/C). E) Individual cell quantification of TDP-43 showed TARDP mutant motor neurons treated with CB5083 results in an increase in the nuclear:cytoplasmic ratio (N/C). Data is collected from 2 independent experimental repeats from 4 CTRL lines (CTRL2, CTRL3, CTRL4, CTRL5), 3 VCP ALS-mutant lines (MUT1, MUT3, MUT4) and 2 TARDBP ALS-mutant lines (MUT5, MUT6). Data is normalised to untreated values for each experimental repeat. Data is plotted as a mean±SD and the p value shown is calculated from an unpaired t-test.

### DETAILED DESCRIPTION OF THE INVENTION

In order for the present invention to be more readily understood, certain terms are first defined below. Additional definitions for the following terms and other terms are set forth throughout the specification.

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%), 6%), 5%, 4%, 3%, 2%, 1%), or less in either direction (greater than or less than) of the stated reference value unless otherwise stated or otherwise evident from the context (except where such number would exceed 100% of a possible value).

As used herein, the term “amelioration” means the prevention, reduction or palliation of a state, or improvement of the state of a subject. Amelioration includes, but does not require, complete recovery or complete prevention of a disease condition.

The term “comparable”, as used herein, refers to a system, set of conditions, effects, or results that is/are sufficiently similar to a test system, set of conditions, effects, or results, to permit scientifically legitimate comparison. Those of ordinary skill in the art will appreciate and understand which systems, sets of conditions, effects, or results are sufficiently similar to be “comparable” to any particular test system, set of conditions, effects, or results as described herein.

The term “correlates”, as used herein, has its ordinary meaning of “showing a correlation with”. Those of ordinary skill in the art will appreciate that two features, items or values show a correlation with one another if they show a tendency to appear and/or to vary, together. In some embodiments, a correlation is statistically significant when its p-value is less than 0.05; in some embodiments, a correlation is statistically significant when its p-value is less than 0.01. In some embodiments, correlation is assessed by regression analysis. In some embodiments, a correlation is a correlation coefficient.

As used herein, the terms “improve,” “increase” or “reduce,” or grammatical equivalents, indicate values that are relative to a reference {e.g., baseline) measurement, such as a measurement taken under comparable conditions {e.g., in the same individual prior to initiation of treatment described herein, or a measurement in a control individual (or multiple control individuals) in the absence of treatment) described herein.

As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids.

A “reference” entity, system, amount, set of conditions, etc., is one against which a test entity, system, amount, set of conditions, etc. is compared as described herein. For example, in some embodiments, a “reference” individual is a control individual who is not suffering from or susceptible to any form of ALS disease; in some embodiments, a “reference” individual is a control individual afflicted with the same form of ALS disease as an individual being treated, and optionally who is about the same age as the individual being treated (to ensure that the stages of the disease in the treated individual and the control individual(s) are comparable).

As used herein, the term “subject”, “individual”, or “patient” refers to any organism upon which embodiments of the invention may be used or administered, e.g., for experimental, diagnostic, prophylactic, and/or therapeutic purposes. Typical subjects include animals {e.g., mammals such as mice, rats, rabbits, non-human primates, and humans; insects; worms; etc.). In a preferred embodiment of the invention the subject is a human.

As used herein, the terms “target cell” or “target tissue” refers to any cell, tissue, or organism that is affected by ALS to be treated, or any cell, tissue, or organism in which a protein involved in ALS is expressed. In some embodiments, target cells, target tissues, or target organisms include those cells, tissues, or organisms in which there is a detectable or abnormally high amount of FUS or TDP-43 {e.g., comparable to that observed in patients suffering from or susceptible to ALS). In some embodiments, target cells, target tissues, or target organisms include those cells, tissues, or organisms that display a disease-associated pathology, symptom, or feature.

As used herein, the phrase “agent” or “therapeutic agent” refers to any agent that, when administered to a subject, has a therapeutic effect and/or elicits a desired biological and/or pharmacological effect. In some embodiments, the therapeutic agent is a VCP inhibitor. In some embodiments, the primary therapeutic agent is a VCP inhibitor which can be used in combination with one or more additional therapeutic agents.

As used herein, the term “therapeutic regimen” refers to any method used to partially or completely alleviate, ameliorate, relieve, inhibit, prevent, delay onset of, reduce severity of and/or reduce incidence of one or more symptoms or features of a particular disease, disorder, and/or condition. It may include administration of one or more doses, optionally spaced apart by regular or varied time intervals. In some embodiments, a therapeutic regimen is one whose performance is designed to achieve and/or is correlated with achievement of {e.g., across a relevant population of cells, tissues, or organisms) a particular effect, e.g., reduction or elimination of a detrimental condition or disease such as ALS. In some embodiments, treatment includes administration of one or more therapeutic agents either simultaneously, sequentially or at different times, for the same or different amounts of time. In some embodiments, a “treatment regimen” includes genetic methods such as gene therapy, gene ablation or other methods known to induce or reduce expression (e.g., transcription, processing, and/or translation of a particular gene product, such as a primary transcript or mRNA).

As used herein, the term “therapeutically effective amount” refers to an amount of a therapeutic agent which confers a therapeutic effect on the treated subject, at a reasonable benefit/risk ratio applicable to any medical treatment. Such a therapeutic effect may be objective (i.e., measurable by some test or marker) or subjective (i.e., subject gives an indication of or feels an effect). In some embodiments, “therapeutically effective amount” refers to an amount of a therapeutic agent or composition effective to treat, ameliorate, or prevent (e.g., delay onset of) a relevant disease or condition, and/or to exhibit a detectable therapeutic or preventative effect, such as by ameliorating symptoms associated with the disease, preventing or delaying onset of the disease, and/or also lessening severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, or on combination with other therapeutic agents. Alternatively or additionally, a specific therapeutically effective amount (and/or unit dose) for any particular patient may depend upon a variety of factors including the particular form of ALS being treated; the severity of the ALS; the activity of the specific therapeutic agent employed; the specific composition employed; the age, body weight, general health, sex and diet of the patient; the time of administration, route of administration, and/or rate of excretion or metabolism of the specific therapeutic agent employed; the duration of the treatment; and like factors as is well known in the medical arts.

As used herein, the term “treatment” (also “treat” or “treating”) refers to any administration of a therapeutic agent according to a therapeutic regimen that achieves a desired effect in that it partially or completely alleviates, ameliorates, relieves, inhibits, delays onset of, reduces severity of and/or reduces incidence of one or more symptoms or features of a particular disease, disorder, and/or condition (e.g., ALS); in some embodiments, administration of the therapeutic agent according to the therapeutic regimen is correlated with achievement of the desired effect. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As used herein, the term neuroprotective agent refers to an agent that prevents or slows the progression of neuronal degeneration and/or prevents neuronal cell death.

### VCP Inhibitory Agents (VCP Inhibitors)

VCP inhibitors may bind to VCP. Binding to VCP polypeptides may be assessed by any technique known to those skilled in the art. Examples of suitable assays include the two hybrid assay system, which measures interactions in vivo, affinity chromatography assays, for example involving binding to polypeptides immobilized on a column, fluorescence assays in which binding of the agent(s) and VCP polypeptides is associated with a change in fluorescence of one or both partners in a binding pair, and the like. Preferred are assays performed in vivo in cells, such as the two-hybrid assay. In a preferred aspect of this embodiment, the invention provides a method for identifying an agent for a pharmaceutical useful in the treatment of ALS, comprising incubating a cell with an agent or agents to be tested and selecting those agents which ameliorate or improve one or more functional parameters associated with ALS.

Examples of agents which are capable of modulating the functional effect of VCP include agents which are inhibitors of VCP and/or VCP adaptor proteins.

VCP inhibitors include the agents mentioned above, as well as the agents shown in Table 1 below, and agents which inhibit and/or disrupt VCP adaptor proteins.

TABLE 1
Exemplary VCP inhibitors

| | Potency of | | | |
|---|---|---|---|---|
| | inhibition (IC50 on | | | |
| | ATPase in vitro, | | | |
| | EC50 on Ub-G76V | VCP domain | Proposed mechanism | |
| Inhibitor | in cell culture | targeted | of inhibiting VCP | References |
| 2-anilino-4-aryl-1,3- | IC50 = 0.07-0.23 μM | D2 ATPase domain | Inhibition of ATPase | [14] |
| thiazoles (several | EC50 = 0.09-1.2 μM | | activity. Reversibility? | |
| distinct compounds) | | | | |
| Syk inhibitor III: 3,4- | IC50 = 1.7 μM | Cys522 in D2 | Inhibition of ATPase | [15] |
| methylenedioxy-6- | | ATPase domain | activity. Irreversible. | |
| nitrostyrene | EC50 = 1.6 μM | | | |
| DBeQ: N²,N⁴- | IC50 = 1.5 μM | D2 ATPase domain | ATP-competitive | [16] |
| dibenzylquinazoline- | | | inhibition of ATPase | |
| 2,4-diamine | EC50 = 2.6 μM | | activity. Reversible | |
| NMS-873 (also | IC50 = 0.03 μM | Not published | Allosteric mechanism | [17] |
| related compound | EC50 = likely to be | | suggested | |
| NMS-859) | around 0.4-0.7 μM | | | |
| Eeyarestatin I | IC50 not | Likely D1 domain | Binding to D1 domain, | [18, 19] |
| | measurable | (D2 and N domains | possibly inducing | |
| | EC50 information: | ruled out as binding | conformational change | |
| | effective inhibition | sites) | and oligomerization of | |
| | at 5-10 μM | | VCP. No effect on | |
| | | | ATPase activity | |
| Xanthohumol | IC50 not | N domain | Binding to N domain. | [20] |
| | determined. EC50 | | | |
| | information: | | | |
| | effective inhibition | | | |
| | around 30 μM | | | |

In some embodiments, the VCP inhibitor is selected from the group consisting of: ML240 (2-(2-Amino-1H-benzimidazole-1-yl)-8-methoxy-N-(phenylmethyl)-4-quinazolinamine), ML241, 2-anilino-4-aryl-1,3-thiazoles, 3,4-methylenedioxy-6-nitrostyrene, DBeQ (N2,N4-dibenzylquinazo-line-2,4-diamine), NMS-873, NMS-859, Eeyarestatin I and Xanthohumol.

In some embodiments, the VCP inhibitor is selected from the group consisting of: ML240 (2-(2-Amino-1H-benzimidazole-1-yl)-8-methoxy-N-(phenylmethyl)-4-quinazolinamine), ML241, 2-anilino-4-aryl-1,3-thiazoles, 3,4-methylenedioxy-6-nitrostyrene, DBeQ (N2,N4-dibenzylquinazo-line-2,4-diamine), CB-5083 (1-[7,8-dihydro-4-[(phenylmethyl)amino]-5H-pyrano[4,3-d]pyrimidin-2-yl]-2-methyl-1H-indole-4-carboxamide), CB-5339 (1-[4-(Benzylamino)-5,6,7,8-tetrahydropyrido[2,3-d]pyrimidin-2-yl]-2-methylindole-4-carboxamide), UPCDC-30245 (1-(3-(5-Fluoro-1H-indol-2-yl)phenyl)-N-(2-(4-isopropylpiperazin-1-yl)ethyl)piperidin-4-amine), NMS-873, NMS-859, Eeyarestatin I and Xanthohumol.

In some embodiments, the VCP inhibitor is selected from the group consisting of: ML240, DBeQ and CB-5083. In some embodiments, the VCP inhibitor is ML240. In some embodiments, the VCP inhibitor is DBeQ. In some embodiments, the VCP inhibitor is CB-5083. In some embodiments, the VCP inhibitor is CB-5083 or CB-5339.

The VCP inhibitors described herein can be used to treat VCP-associated or non-VCP associated ALS. Preferably, the ALS is non-VCP-associated ALS and the VCP inhibitor inhibits the D2 ATPase domain of VCP. Accordingly, in a preferred aspect, the invention provides a VCP inhibitor for use in a method of treating or preventing non-VCP-associated ALS in a subject, wherein the VCP inhibitor inhibits the D2 ATPase domain of VCP. For example, in some embodiments, the ALS is non-VCP-associated ALS and the VCP inhibitor is CB-5083 or CB-5339. In some embodiments, the subject has been identified as not having a disease-causing mutation in a VCP gene and the VCP inhibitor inhibits the D2 ATPase domain of VCP. In some embodiments, the subject has been identified as not having a disease-causing mutation in a VCP gene and the VCP inhibitor is CB-5083 or CB-5339.

In some embodiments, the subject has, or has been identified as having, one or more disease-causing genetic mutations in a TARDBP gene and the VCP inhibitor inhibits the D2 ATPase domain of VCP. In some embodiments, the subject has, or has been identified as having, one or more disease-causing genetic mutations in a TARDBP gene and the VCP inhibitor is CB-5083 or CB-5339. In some embodiments, the subject has, or has been identified as having, a disease-causing genetic mutation in a TARDBP gene at position G298 and the VCP inhibitor inhibits the D2 ATPase domain of VCP. In some embodiments, the mutation at position G298 is a G298S mutation. In some embodiments, the subject has, or has been identified as having, a disease-causing genetic mutation in a TARDBP gene at position G298 and the VCP inhibitor is CB-5083 or CB-5339. In some embodiments, the mutation at position G298 is a G298S mutation.

VCP adaptor proteins are known in the art. For example, see [21], especially Table 1 therein. Moreover, methods are known for identifying VCP adaptor proteins. For example [22] describes a method based on unbiased mass spectrometry, which they use to identify a complex between VCP and the UBXD1 cofactor.

Agents which influence the activity or localisation of VCP may be of almost any general description, including low molecular weight agents, including organic agents which may be linear, cyclic, polycyclic or a combination thereof, peptides, polypeptides including antibodies, or proteins. In general, as used herein, “peptides”, “polypeptides” and “proteins” are considered equivalent. Certain VCP inhibitors are set forth above in table 1. See also [23] for examples of other useful VCP inhibitors.

As used herein, a “VCP inhibitor” is a drug which is capable of inhibiting the activity of VCP which is required for normal neuronal cell function. Inhibitors of VCP are known in the art and are being discovered regularly, as VCP is also a target for cancer therapy and other medical disciplines. Exemplary inhibitors include those described above and methods for identifying VCP inhibitors are described in the prior art. In some embodiments, VCP inhibitors of the invention can inhibit the activity of VCP by inhibiting the D2 ATPase domain of VCP.

A VCP inhibitor may be referred to as an VCP antagonist.

### Pharmaceutical Compositions of the Agent

The agent can be in the form of a pharmaceutical composition. The pharmaceutical composition can comprise the agent (i.e. the VCP inhibitor). The pharmaceutical compositions can comprise about 5 nanograms (ng) to about 10 milligrams (mg) of the agent. In some embodiments, pharmaceutical compositions according to the present invention comprise about 25 ng to about 5 mg of the agent. In some embodiments, the pharmaceutical compositions contain about 50 ng to about 1 mg of the agent. In some embodiments, the pharmaceutical compositions contain about 0.1 to about 500 micrograms of the agent. In some embodiments, the pharmaceutical compositions contain about 1 to about 350 micrograms of the agent. In some embodiments, the pharmaceutical compositions contain about 5 to about 250 micrograms of the agent. In some embodiments, the pharmaceutical compositions contain about 10 to about 200 micrograms of the agent. In some embodiments, the pharmaceutical compositions contain about 15 to about 150 micrograms of the agent. In some embodiments, the pharmaceutical compositions contain about 20 to about 100 micrograms of the agent. In some embodiments, the pharmaceutical compositions contain about 25 to about 75 micrograms of the agent. In some embodiments, the pharmaceutical compositions contain about 30 to about 50 micrograms of the agent. In some embodiments, the pharmaceutical compositions contain about 35 to about 40 micrograms of the agent. In some embodiments, the pharmaceutical compositions contain about 100 to about 200 micrograms of the agent. In some embodiments, the pharmaceutical compositions comprise about 10 micrograms to about 100 micrograms of the agent. In some embodiments, the pharmaceutical compositions comprise about 20 micrograms to about 80 micrograms of the agent. In some embodiments, the pharmaceutical compositions comprise about 25 micrograms to about 60 micrograms of the agent. In some embodiments, the pharmaceutical compositions comprise about 30 ng to about 50 micrograms of the agent. In some embodiments, the pharmaceutical compositions comprise about 35 ng to about 45 micrograms of the agent. In some embodiments, the pharmaceutical compositions contain about 0.1 to about 500 micrograms of the agent. In some embodiments, the pharmaceutical compositions contain about 1 to about 350 micrograms of the agent. In some embodiments, the pharmaceutical compositions contain about 25 to about 250 micrograms of the agent. In some embodiments, the pharmaceutical compositions contain about 100 to about 200 micrograms of the agent.

In other embodiments, the pharmaceutical composition can comprise up to and including 15, 20, 25, 30, 35, 40, 45, 50, 55, 60, 65, 70, 75, 80, 85, 90, 95 or 100 ng of the agent. In some embodiments, the pharmaceutical composition can comprise up to and including 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995, or 1000 micrograms of the agent. In some embodiments, the pharmaceutical composition can comprise up to and including 1.5, 2, 2.5, 3, 3.5, 4, 4.5, 5, 5.5, 6, 6.5, 7, 7.5, 8, 8.5, 9, 9.5 or 10 mg of the agent.

In some embodiments, the dose of an agent of the invention is from about 0.5 .mg and about 5,000 mg. In some embodiments, a dose of an agent of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second agent as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the agents of the invention are administered to the patient in dosages that range from one to five times per day or more. In another embodiment, the agents of the invention are administered to the patient in a range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physical taking all other factors about the patient into account.

The pharmaceutical composition can further comprise other agents for formulation purposes according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are suitable. Stabilizers include gelatin and albumin.

The agent can further comprise a pharmaceutically acceptable excipient. The pharmaceutically acceptable excipient can be functional molecules such as vehicles, adjuvants, carriers, or diluents.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

For oral application, particularly suitable are tablets, dragees, liquids, drops, suppositories, or capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients which are suitable for the manufacture of tablets. Such excipients include, for example, an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Tablets may further comprise a sweetening agent, a flavoring agent, a coloring agent, a preservative, or some combination of these in order to provide for a palatable preparation.

### Controlled Release Formulations and Drug Delivery Systems

Controlled- or sustained-release formulations of a pharmaceutical composition of the invention may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time.

Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of the drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example, pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed-release and pulsatile release formulations.

The term sustained release is used in its conventional sense to refer to a drug formulation that provides for a gradual release of a drug over an extended period of time, and that may, although not necessarily, result in substantially constant blood levels of a drug over an extended time period. The period of time may be as long as a month or more and should be a release which is longer than the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation. In a preferred embodiment of the invention, the compounds of the invention are administered to a patient, alone or in combination with another pharmaceutical agent, using a sustained release formulation.

The term delayed-release is used herein in its conventional sense to refer to a drug formulation that provides for an initial release of the drug after some delay following drug administration and that includes a delay of from about 10 minutes up to about 12 hours.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Kits

An agent described herein can be provided in a kit. In some instances, the kit includes (a) a container that contains an agent described herein and, optionally (b) informational material. The informational material can be descriptive, instructional, marketing or other material that relates to the methods described herein and/or the use of an agent, e.g., for therapeutic benefit.

The informational material of the kits is not limited in its form. In some instances, the informational material can include information about production of a therapeutic agent, molecular weight of a therapeutic agent, concentration, date of expiration, batch or production site information, and so forth. In other situations, the informational material relates to methods of administering a therapeutic agent, e.g., in a suitable amount, manner, or mode of administration (e.g., a dose, dosage form, or mode of administration described herein). The method can be a method of treating a subject having ALS.

In some cases, the informational material, e.g., instructions, is provided in printed matter, e.g., a printed text, drawing, and/or photograph, e.g., a label or printed sheet. The informational material can also be provided in other formats, such as Braille, computer readable material, video recording, or audio recording. In other instances, the informational material of the kit is contact information, e.g., a physical address, email address, website, or telephone number, where a user of the kit can obtain substantive information about a therapeutic agent therein and/or their use in the methods described herein. The informational material can also be provided in any combination of formats.

In addition to a therapeutic agent, the kit can include other ingredients, such as a solvent or buffer, a stabilizer, or a preservative. The kit can also include further agents, e.g., a second or third agent, e.g., other therapeutic agents. The components can be provided in any form, e.g., liquid, dried or lyophilized form. The components can be substantially pure (although they can be combined together or delivered separate from one another) and/or sterile. When the components are provided in a liquid solution, the liquid solution can be an aqueous solution, such as a sterile aqueous solution. When the components are provided as a dried form, reconstitution generally is by the addition of a suitable solvent. The solvent, e.g., sterile water or buffer, can optionally be provided in the kit.

The kit can include one or more containers for a therapeutic agent or other agents. In some cases, the kit contains separate containers, dividers or compartments for a therapeutic agent and informational material. For example, a therapeutic agent can be contained in a bottle, vial, or syringe, and the informational material can be contained in a plastic sleeve or packet. In other situations, the separate elements of the kit are contained within a single, undivided container. For example, a therapeutic agent can be contained in a bottle, vial or syringe that has attached thereto the informational material in the form of a label. In some cases, the kit can include a plurality (e.g., a pack) of individual containers, each containing one or more unit dosage forms (e.g., a dosage form described herein) of a therapeutic agent. The containers can include a unit dosage, e.g., a unit that includes a therapeutic agent. For example, the kit can include a plurality of syringes, ampules, foil packets, blister packs, or medical devices, e.g., each containing a unit dose. The containers of the kits can be air tight, waterproof (e.g., impermeable to changes in moisture or evaporation), and/or light-tight.

The kit can optionally include a device suitable for administration of a therapeutic agent, e.g., a syringe or other suitable delivery device. The device can be provided preloaded with a therapeutic agent, e.g., in a unit dose, or can be empty, but suitable for loading.

### Amyotrophic Lateral Sclerosis (ALS)

Amyotrophic lateral sclerosis (ALS) is an adult-onset, fatal neurodegenerative disorder, characterized by degeneration of both upper motor neurons in the primary motor cortex and lower motor neurons in the brainstem and spinal cord. Symptoms of ALS initially include muscle atrophy and weakness. Subsequently, spreading paralysis of the voluntary muscles, and eventually the respiratory muscles, often develops. Approximately 50% of patients with ALS die within 30 months of symptom onset, often from respiratory insufficiency, whereas about 10% of patients may survive for more than a decade [24].

About 10%-15% of ALS patients have a familial form of the disease, with at least two first-degree or second-degree relatives with ALS [25]. If no family history is identified, the diagnosis is assumed to be sporadic (or non-familial). The incidence of sporadic ALS shows little variation in the Western countries, ranging from 1 to 2 per 100,000 person-years, with an estimated lifetime risk of 1 in 400. ALS is rare before the age of 40 years and increases exponentially with age thereafter. Mean age at onset is 58-63 years for sporadic ALS and 40-60 years for familial ALS, with a peak incidence in those aged 70-79 years. Men have a higher risk of ALS than women, leading to a male-to-female ratio of 1.2-1.5 [24].

In some embodiments, ALS can be familial ALS. In some embodiments, ALS can be sporadic ALS. In some embodiments familial ALS is defined as a patient having more than one occurrence of the disease in a family history. In some embodiments sporadic ALS is defined as a patient having no known history of other family members with the disease. In some embodiments, ALS can be associated with one or more disease-causing genetic mutation mutations in the VCP protein. In some embodiments ALS can be familial ALS associated with one or more disease-causing genetic mutation mutations in the VCP protein. In some embodiments ALS can be sporadic ALS associated with one or more disease-causing genetic mutation mutations in the VCP protein.

In some embodiments, the subject has one or more disease-causing genetic mutations in a gene other than a VCP gene. The disease-causing mutations may be known disease-causing mutations. The disease-causing mutations may be ALS-causing mutations. For example, in some embodiments, the subject has one or more disease-causing genetic mutations in a TARDBP gene. Thus, the ALS may, in some embodiments, be associated with one or more genetic mutations in a TARDBP gene. In some embodiments, the subject has been identified as having one or more disease-causing genetic mutations in a TARDBP gene. Such mutations will be known in the art. In some embodiments, the subject has, or has been identified as having, a disease-causing genetic mutation in a TARDBP gene at any one or more of positions S292, G294, G295, G298, A315, A382, M337, G348 or S393. In some embodiments, the subject has, or has been identified as having, one or more disease-causing genetic mutations in a TARDBP gene selected from the list consisting of S292N, G294V, G295S, G298S, A315T, A382T, M337V, G348C and S393L. In some embodiments, the subject has, or has been identified as having, a disease-causing genetic mutation in a TARDBP gene at position G298. In some embodiments, the mutation at position G298 is a G298S mutation.

### Disease-Causing Genetic Mutations in VCP

ALS subjects can be identified as having one or more known disease-causing mutations in a VCP gene. Such subjects can be characterised as having VCP-associated ALS. ALS subjects can be identified as not having one or more known disease-causing mutations in a VCP gene. Such subjects can be characterised as having non-VCP-associated ALS. The present invention provides a method of treating or preventing ALS in subjects that have not been identified as having a disease-causing mutation in a VCP gene. The present invention also provides a method of treating or preventing ALS in subjects that have been identified as not having a disease-causing mutation in a VCP gene. The present invention also provides a VCP inhibitor for use in a method of treating or preventing ALS in subjects that have not been identified as having a disease-causing mutation in a VCP gene. The present invention also provides a VCP inhibitor for use in methods of treating or preventing ALS in subjects that have been identified as not having a disease-causing mutation in a VCP gene.

In some embodiments the subject has been identified as not having any of the VCP disease-causing genetic mutations listed in Table 2. In some embodiments the subject has been identified as not having a disease-causing genetic mutation in a VCP gene at any of positions R95, I114, I151, R155, G156, M158, R159, R191, N387, N401, R487, D592, R662 and N750. In some embodiments, the patient has been identified as not having a disease-causing genetic mutation in a VCP gene selected from the list consisting of: R95C, R95G, I114V, I151V, R155H, R155C, G156C, M158V, R159G, R159C, R159H, R191G, R191Q, N387T, N401S, R487H, D592N, R662C and N750S.

An estimated 5 to 10 percent of ALS is familial and caused by mutations in one of several genes. The pattern of inheritance varies depending on the gene involved. Most cases are inherited in an autosomal dominant pattern, which means one copy of the altered gene in each cell is sufficient to cause the disorder. In most cases, an affected person has one parent with the condition. Some people who inherit a familial genetic mutation known to cause ALS never develop features of the condition. It is unclear why some people with a mutated gene develop the disease and other people with a mutated gene do not.

Less frequently, ALS is inherited in an autosomal recessive pattern, which means both copies of the gene in each cell have mutations. The parents of an individual with an autosomal recessive condition each carry one copy of the mutated gene, but they typically do not show signs and symptoms of the condition. Because an affected person's parents are not affected, autosomal recessive ALS is often mistaken for sporadic ALS even though it is caused by a familial genetic mutation.

Very rarely, ALS is inherited in an X-linked dominant pattern. X-linked conditions occur when the gene associated with the condition is located on the X chromosome, which is one of the two sex chromosomes. In females (who have two X chromosomes), a mutation in one of the two copies of the gene in each cell is sufficient to cause the disorder. In males (who have only one X chromosome), a mutation in the only copy of the gene in each cell causes the disorder. In most cases, males tend to develop the disease earlier and have a decreased life expectancy compared with females. A characteristic of X-linked inheritance is that fathers cannot pass X-linked traits to their sons.

About 90 to 95 percent of ALS cases are sporadic, which means they are not inherited.

In both sporadic and familial ALS, the patients may have one or more disease-causing genetic mutations in the VCP gene. Several disease-causing mutations in the VCP gene are well-characterised in the prior art and the skilled person is aware of suitable gene panels which can be used to identify patients carrying disease-causing mutations. Table 2 indicates a non-exhaustive list of a number of known disease-causing mutations in the VCP gene.

TABLE 2
List of known disease-causing VCP mutations

| | Change in | Change in | Location in | |
|---|---|---|---|---|
| Position | amino acid | gene | protein | Phenotype |
| R95 | R95C | 283C > T | N domain | IBM, ALS |
| | R95G | 283C > G | N domain | IBM, PDB, FTD, |
| | | | | ALS |
| I114 | I114V | 340A > G | N domain | ALS |
| I151 | I151V | 451A > G | N domain | IBM, ALS |
| | R155H | 464G > A | N domain | IBM, PDB, FTD, |
| | | | | ALS |
| | R155C | 463C > T | N domain | IBM, PDB, FTD, |
| | | | | ALS |
| G156 | G156C | 466G > C | N domain | ALS |
| M158 | M158V | 472A > G | N domain | PDB, ALS |
| R159 | R159G | 475C > G | N domain | ALS, FTD |
| | R159C | 475C > T | N domain | IBM, FTD, PD, ALS |
| | R159H | 476G > A | N domain | IBM, PDB, FTD, |
| | | | | ALS |
| R191 | R191G | 571C > G | N-D1 linker | IBM, ALS |
| | R191Q | 572G > A | N-D1 linker | IBM, PDB, FTD, |
| | | | | ALS |
| | N387T | 1160A > C | D1 domain | ALS |
| N401 | N401S | 1202A > G | D1 domain | FTD, ALS |
| R487 | R487H | 1460G > A | D2 domain | FTD, ALS |
| D592 | D592N | 1774G > A | D2 domain | ALS |
| R662 | R662C | 1984C > T | D2 domain | ALS |
| N750 | N750S | 2249A > G | D2 domain | ALS |

### Methods of Diagnosis

In order to establish whether a subject has any disease-causing mutations in a VCP gene, the invention also encompasses methods of diagnosing a subject as having non-VCP-associated ALS. Embodiments of the invention may therefore include determining whether a subject has any disease-causing mutations in a VCP gene. Several appropriate methods will be known by the skilled person for establishing the sequence of a VCP gene in a biological sample from the subject. In some embodiments, the determining whether a subject has any disease-causing mutations in a VCP gene comprises the step of establishing the sequence of a VCP gene, wherein the sequence of a VCP gene is established using any one or more of the following techniques: DNA sequencing, RNA sequencing, microarray analysis, real time quantitative PCR, Northern blot analysis, in situ hybridisation and/or detection and quantification of a binding molecule. In a preferred embodiment, establishing whether a subject has any disease-causing mutations in a VCP gene comprises use of DNA sequencing. A decision to provide a treatment to the subject, or to provide a recommendation of a treatment to the subject, may be made on the basis of determining the presence of one or more disease-causing mutations in a VCP gene. Recommendations to provide a treatment may be provided in the form of a report. Embodiments of the invention may therefore comprise a step of providing a report, wherein the report comprises information on the presence or absence in the subject of one or more disease-causing mutations in a VCP gene. The report may additionally or alternatively include a recommendation to provide a treatment to the subject (such as a VCP inhibitor) for example on the basis of the presence of one or more disease-causing mutations in a VCP gene, or a recommendation to not provide a treatment to the subject (such as a VCP inhibitor) for example on the basis of the absence of one or more disease-causing mutations in a VCP gene.

A step of determining whether a subject has one or more disease-causing mutations may be performed on a sample from the subject. The method may comprise the step of obtaining said sample from the subject, or the sample may have been obtained from the subject at an earlier point in time. Sample may include, for example, a plasma or blood sample.

Treating ALS with Agents of the Invention

In some embodiments, an agent is provided to the central nervous system of a subject, e.g., a subject suffering from or susceptible to ALS. In some embodiments, an agent is provided to one or more of target cells or tissues of brain, spinal cord, and/or peripheral organs. In some embodiments, target cells or tissues include those cells or tissues that display a disease-associated pathology, symptom, or feature. In some embodiments, target cells or tissues include those cells or tissues in which TDP-43 or FUS/TLS is expressed at an elevated level, e.g., cells in which TDP-43 or FUS/TLS is expressed at an elevated level in the cytoplasm of the cells. As used herein, a target tissue may be a brain target tissue, a spinal cord target tissue and/or a peripheral target tissue.

Compositions described herein can be provided directly into the CNS of a subject suffering from or at risk of developing ALS, thereby achieving a therapeutic concentration within the affected cells and tissues of the CNS (e.g., the brain). For example, one or more agents can be provided to target cells or tissues of the brain, spinal cord and/or peripheral organs to treat ALS. As used herein, the term “treat” or “treatment” refers to amelioration of one or more symptoms associated with the disease, prevention or delay of the onset of one or more symptoms of ALS.

In some embodiments, treatment refers to partially or complete alleviation, amelioration, relief, inhibition, delaying onset, reducing severity and/or incidence of neurological impairment in a patient suffering from or susceptible to ALS. As used herein, the term “neurological impairment” includes various symptoms associated with impairment of the central nervous system {e.g., the brain and spinal cord). Symptoms of neurological impairment may include, for example, developmental delay, progressive cognitive impairment, hearing loss, impaired speech development, deficits in motor skills, hyperactivity, aggressiveness and/or sleep disturbances, among others.

In some embodiments, treatment refers to decreased toxicity of various cells or tissues. In some embodiments, treatment refers to decreased neuronal toxicity due to FUS or TDP-43 in brain target tissues, spinal cord neurons, and/or peripheral target tissues. In certain embodiments, toxicity is decreased by about 5%, 10%, 15%, 20%, 25%, 30%, 35%, 40%, 45%, 50%, 55%, 60%, 65%, 70%, 75%, 80%, 85%, 90%, 95%, 100% or more as compared to a control. In some embodiments, toxicity is decreased by at least 1-fold, 2-fold, 3-fold, 4-fold, 5-fold, 6-fold, 7-fold, 8-fold, 9-fold or 10-fold as compared to a control. In some embodiments, toxicity is measured by tests known to those of ordinary skill in the art including, but not limited to, neuroimaging methods (e.g., CT scans, MRI, functional MRI, etc.).

In certain embodiments, treatment according to the present disclosure results in a reduction (e.g., about a 5%, 10%, 15%, 20%, 25%, 30%, 40%, 50%, 55%, 60%, 65%, 70%, 75%, 80%, 90%, 95%, 97.5%, 99% or more reduction) or a complete elimination of the presence, or alternatively the accumulation, of one or more pathological, clinical, or biological markers that are associated with ALS. For example, in some embodiments, upon administration to a subject, a pharmaceutical composition described herein demonstrates or achieves a reduction in muscle loss, muscle twitching, muscle weakness, spasticity, abnormal tendon reflexes, Babinski sign, breathing problems, facial weakness, slurred speech, loss of perception, loss of reasoning, loss of judgment, and/or loss of imagination.

In some embodiments, treatment refers to increased survival (e.g., survival time). For example, treatment can result in an increased life expectancy of a patient. In some embodiments, treatment results in an increased life expectancy of a patient by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 1 10%, about 1 15%, about 120%, about 125%, about 130%, about 135%, about 140%, about 145%, about 150%, about 155%, about 160%, about 165%, about 170%, about 175%, about 180%, about 185%, about 190%, about 195%, about 200% or more, as compared to the average life expectancy of one or more control individuals with ALS without treatment. In some embodiments, treatment results in an increased life expectancy of a patient by more than about 6 months, about 7 months, about 8 months, about 9 months, about 10 months, about 1 1 months, about 12 months, about 2 years, about 3 years, about 4 years, about 5 years, about 6 years, about 7 years, about 8 years, about 9 years, about 10 years or more, as compared to the average life expectancy of one or more control individuals with ALS without treatment. In some embodiments, treatment results in long term survival of a patient. As used herein, the term “long term survival” refers to a survival time or life expectancy longer than about 40 years, 45 years, 50 years, 55 years, 60 years, or longer.

The term “improve,” “increase” or “reduce,” as used herein, indicates values that are relative to a control. In some embodiments, a suitable control is a baseline measurement, such as a measurement in the same individual prior to initiation of the treatment described herein, or a measurement in a control individual (or multiple control individuals) in the absence of the treatment described herein. A “control individual” is an individual afflicted with ALS, who is about the same age and/or gender as the individual being treated (to ensure that the stages of the disease in the treated individual and the control individual(s) are comparable).

In some embodiments, the disease-causing genetic mutation is associated with loss of VCP-dependent endocytic mechanisms of cytoplasmic proteostasis. In some embodiments, the disease-causing genetic mutation is associated with mislocalization of RBPs (such as TDP-43, FUS and/or SFPQ). In some embodiments the disease-causing genetic mutation is associated with reduction in the nuclear-to-cytoplasmic ratios of one or more of TDP-43, FUS and/or SFPQ. In some embodiments the disease-causing genetic mutation is associated with reduction in the nuclear-to-cytoplasmic ratio of TDP-43. In some embodiments the disease-causing genetic mutation is associated with reduction in the nuclear-to-cytoplasmic ratio of FUS. In some embodiments the disease-causing genetic mutation is associated with reduction in the nuclear-to-cytoplasmic ratio of SFPQ. In some embodiments the disease-causing genetic mutation is associated with one or more (e.g. 1, 2, 3, 4, 5 or more) of the above mentioned normal functions of VCP. In some embodiments the disease-causing genetic mutation is associated with one or more point mutations in the VCP protein. In some embodiments the disease-causing genetic mutation is associated with mutations at one or more positions selected from the list consisting of: R95, I114, I151, R155, G156, M158, R159, R191, N387, N401, R487, D592, R662 and N750. In some embodiments the disease-causing genetic mutation is associated with one or more mutations selected from the list consisting of: R95C, R95G, I114V, I151V, R155H, R155C, G156C, M158V, R159G, R159C, R159H, R191G, R191Q, N387T, N401S, R487H, D592N, R662C and N750S.

The term “associated with” is used herein to describe an observed correlation between two items or events. For example, a disease-causing genetic mutation in VCP may be considered to be “associated with” a particular neurological dysfunction or disorder if its presence or level correlates with a presence or level of the dysfunction or disorder.

The individual (also referred to as “patient” or “subject”) being treated is an individual (fetus, infant, child, adolescent, or adult human) having ALS or having the potential to develop ALS. In some instances, a subject to be treated is genetically predisposed to developing ALS. For example, a subject to be treated may have a mutation in a VCP gene, SOD1 gene, ALS2 gene, VAPB gene, SETX gene, TDP-43 gene, FUS/TLS gene, and/or OPTN gene. In In some embodiments the patient has no genetic predisposition to developing ALS. In some embodiments, a subject to be treated may have no known disease-causing mutations in a VCP gene, SOD1 gene, ALS2 gene, VAPB gene, SETX gene, TDP-43 gene, FUS/TLS gene, and/or OPTN gene. In a preferred embodiment, a subject to be treated may have no known disease-causing mutations in a VCP gene.

### Combination Therapies

In some embodiments, an agent (such as a VCP inhibitor) described herein is administered to a subject in combination with one or more additional therapies to treat ALS or one or more symptoms of ALS. For example, an agent can be administered in combination with riluzole, baclofen, diazepam, trihexyphenidyl or amitriptyline.

In some embodiments, combined administration of a first agent (such as a VCP inhibitor) and a second agent results in an improvement in ALS or a symptom thereof to an extent that is greater than one produced by either the first agent or the second agent alone. The difference between the combined effect and the effect of each agent alone can be a statistically significant difference.

In some embodiments, combined administration of a first agent and a second agent allows administration of the second agent at a reduced dose, at a reduced number of doses, and/or at a reduced frequency of dosage compared to a standard dosing regimen approved for the second agent.

In some embodiments, an immunosuppressant agent known to the skilled artisan can be administered to a subject in combination with an agent described herein. Exemplary immunosuppressant agents include, without limitation, cyclosporine, FK506, rapamycin, CTLA4-Ig, anti-TNF agents (such as etanercept), daclizumab (e.g., Zenapax™), anti-CD2 agents, anti-CD4 agents, and anti-CD40 agents.

### Routes of Administration

The agent or pharmaceutical composition can be administered by different routes including orally, parenterally, sublingually, transdermally, rectally, transmucosally, topically, via inhalation, via buccal administration, intrapleurally, intravenously, intraarterially, intraperitoneally, subcutaneously, intramuscularly, intranasal intrathecally, and/or intraarticularly, or combinations thereof. In some embodiments the agent or pharmaceutical composition is administered orally.

The present invention is further illustrated in the following Examples. It should be understood that these Examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims. Furthermore, features of each aspect of the invention are as for each of the other aspects mutatis mutandis. For example, embodiments relating to types of VCP inhibitors, disease causing mutations, type of ALS etc., that are provided in the context of the VCP inhibitor for use according to the invention, apply equally to the methods of diagnosis, compositions, and kits of the invention.

### EXAMPLES

### Example 1—Materials and Methods

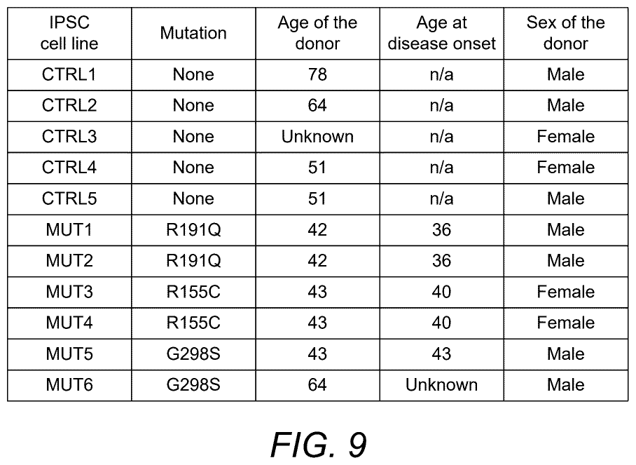

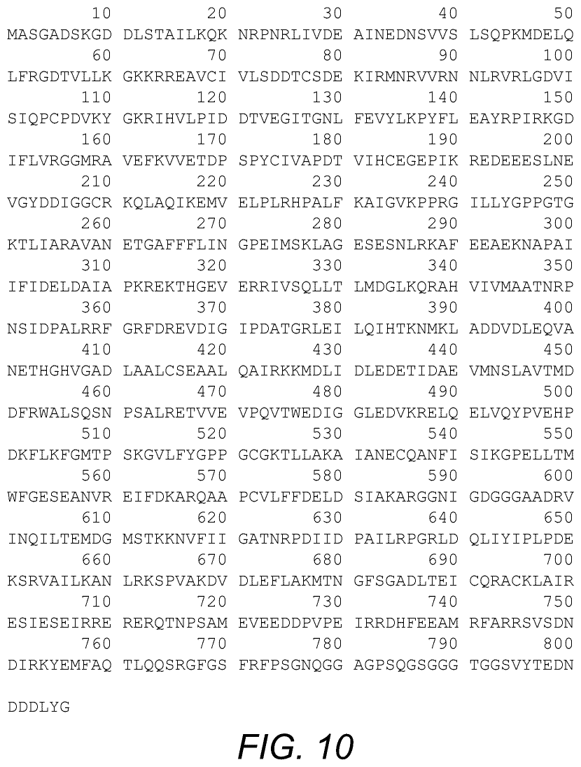

Human fibroblasts and iPSC. Dermal fibroblasts were cultured in OptiMEM+10% FCS medium. For iPSC generation, episomal plasmids, pCXLE hOct4 shp53, pCXLE hSK, and pCXLE hUL (Addgene) [26], were transfect into dermal fibroblasts. Three control lines used are commercially obtainable (control 2, control 3 and control 5) and were purchased from Coriell (cat. Number ND41866*C), ThermoFisher Scientific (cat. number A18945) and Cedars-Sinai (CS02iCTR-NTn4). TARDBP mutant lines (MUT5 and MUT6) used are commercially available and were purchased from NINDS (ND50007) and Cedars-Sinai (CS47i). Details of the iPSC lines used in the study can be found in FIG. 9.

Cell culture and motor neuron differentiation. IPSCs were maintained with Essential 8 Medium media (Life Technologies) on Geltrex (Life Technologies), and passaged using EDTA (Life Technologies, 0.5 mM). IPSC cultures were kept at 37° C. and 5% carbon dioxide. IPSCs underwent differentiation into spinal cord motor neurons as described in Hall et al, 2017 [12].

IPSC were plated to 100% confluency and then differentiated to the neuroepithelium in medium consisting of DMEM/F12 Glutamax, Neurobasal, L-Glutamine, N2 supplement, nonessential amino acids, B27 supplement, β-mercaptoethanol (all from Life Technologies) and insulin (Sigma). The cells underwent a sequential treatment with small molecules, with day 0-7: 1 μM Dorsomorphin (Millipore), 2 μM SB431542 (Tocris Bioscience), and 3.3 μM CHIR99021 (Miltenyi Biotec), day 7-14: 0.5 μM retinoic acid (Sigma) and 1 μM Purmorphamine (Sigma), day 14-18: 0.1 μM Purmorphamine. Following 18 days of neural conversion and patterning, neural precursors were terminally differentiated in 0.1 μM Compound E (Enzo Life Sciences).

Throughout the neuroepithelial layer was enzymatically dissociated using dispase (GIBCO, 1 mg ml−1). The neural precursors were dissociated with Accutase (Life Technologies) for final plating onto a 96 well plate (Falcon) coated with polyethylenimine (PEI) (2.2 mg/ml in 0.1M of sodium borate (Sigma) and Geltrex. Following 6 days of terminal differentiation, cells were fixed in 4% paraformaldehyde for immunolabeling.

Inhibitor treatment. Motor neuron cultures were treated with 1 μM of ML240 (Sigma; SML1071; CAS:1346527-98-7) for 2 hours, 5 μM of DBeQ for 3 hours or 1 μM of CB-5083 for 3 hours.

Immunofluorescence staining. Cells were fixed in 4% paraformaldehyde in PBS for 15 minutes at room temperature (RT). For permeabilization and non-specific antibody blocking, 0.3% Triton-X containing 5% bovine serum albumin (BSA) (Sigma) in PBS was added for 60 minutes. Primary antibodies were made up in 5% BSA and then applied overnight at 4° C. Primary antibodies used were SMI-32 (BioLegend; 801701; mouse; 1:1000), ChAT (Millipore; AB144P; goat; 1:100), βIII-tubulin (abcam; ab41489; chicken; 1:1000), TDP-43 (ProteinTech; 12892-1-AP; rabbit; 1:400), SFPQ (abcam; ab11825; mouse; 1:400), FUS (Santa Cruz; sc-47711; mouse; 1:200), hnRNPA1 (Cell Signaling; 8443S; rabbit; 1:500), hnRNPK (Santa Cruz; sc-28380; mouse; 1:500). A species-specific Alexa Fluor-conjugated secondary antibody (Life Technologies) at 1:1000 dilution in 5% BSA was added for 90 minutes at RT in the dark. Cells were washed once in PBS containing DAPI, 4′,6-diamidino-2-phenylindole nuclear stain (1:1000) for 10 minutes.

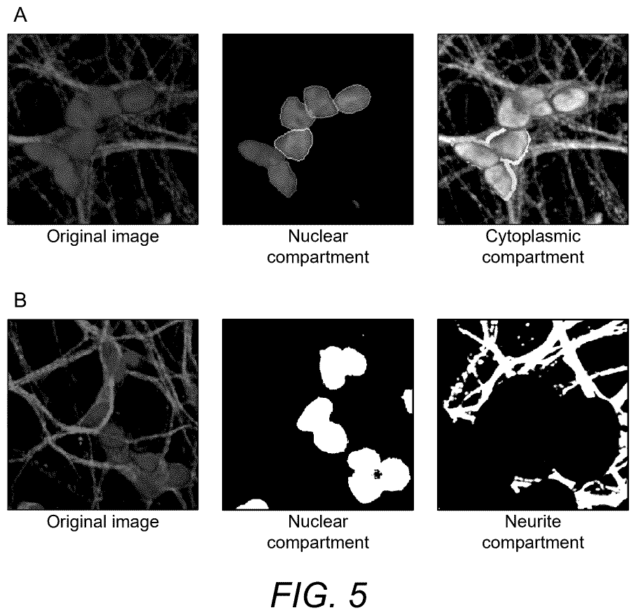

Image acquisition and analysis. Images were acquired using the Opera Phenix High-Content Screening System (Perkin Elmer). Images were acquired with a 40× objective as confocal z-stacks with a z-step of 1 μm. Stacks were processed to obtain a maximum intensity projection. A minimum of 12 fields of view were taken for each well. To calculate the nuclear:cytoplasmic ratio of RNA-binding proteins (RBPs) in single cells, images were analysed using the Columbus Image Analysis System (Perkin Elmer). A DAPI mask defined the nucleus, and based on nuclear properties a trained machine learning feature selected neurons in an automated fashion. For each individual cell an average nuclear intensity of the RBP of interest was measured. For the cytoplasmic measurement, a 1.5 μm cytoplasmic region was defined around the nucleus within a cytoplasmic mask and an average intensity measured. An example of this nuclear and cytoplasmic compartments defined by this analysis can be found in FIG. 5A. A ratio of the nuclear:cytoplasmic average intensity measurements was calculated per cell. An average of each field was calculated and then averaged across the well.

For the nuclear:neurite ratio, we implemented a semi-automated image analysis pipeline combining Ilastik [27], Cellprofiler [28] and ImageJ. Nuclear segmentation was performed using DAPI stained images, in which intensity was scaled in ImageJ from 0-500 to allow improved nuclear detection. A randomly selected subset of images was used in Ilastik for generation of a binary nuclear segmentation mask. To define the neurite compartment βIII-tubulin was used to create a neuronal mask as it is a reliable axonal and dendritic marker. To remove the nuclei and cytoplasm from the βIII-tubulin mask the nuclei were expanded by 30 pixels and removed, which ensured only the neurites were included in the analysis. An example of the compartments defined by this analysis can be found in FIG. 5B. Intensity measurements of the protein of interest were performed in Cellprofiler using the nuclear, and neurite masks. Calculation of intensity values and ratios were performed using custom R scripts.

When examining the nuclear:cytoplasmic or nuclear:neurite ratio, if an increase or decrease in the ratio is detected, it is not apparent which cellular compartment is contributing to the change. To address this for the nuclear:neurite ratio we have utilised the presence of compartment specific markers. The same semi-automated image analysis pipeline was implemented as for the nuclear:neurite ratio as described above, but in addition to the protein of interest, intensity measurements were performed for DAPI and βIII-tubulin and used to calculate the specified ratio.

Western blot analysis. Protein levels of TDP-43, FUS and SFPQ were assessed in whole cells in control and VCP mutant motor neurons. Cells were subjected to untreated conditions or treatment of 1 μM of ML240 for 2 hours prior to protein extraction. The cells were lysed and proteins were extracted with RIPA disruption. Total protein concentration was quantified using BCA protein assay (Sigma). Equal amounts of protein samples were then loaded onto a gel and separated by SDS PAGE and transferred onto a nitrocellulose membrane. Samples were then blocked with PBS, 0.1% Tween, 5% dry milk powder at RT for one hour followed by primary antibody incubation overnight at 4° C. The following antibodies were diluted in PBS 5% BSA; TDP-43 (ProteinTech; 12892-1-AP; rabbit; 1:1000), SFPQ (Abcam; 11825; mouse; 1:250), FUS (Santa Cruz; sc-47711; mouse; 1:500), GAPDH (GeneTex; GT239; mouse; 1:10000). For detection, membranes were incubated with species-specific near infra-red fluorescent antibodies (IRDye, Licor) at RT for one hour and imaged using an Odyssey Fc Imaging System (Licor).

Statistical analysis. There are 3 control and 4 VCP mutant iPSC lines, with details found in FIG. 9. The number of cells used in each experiment is stated within the figure legends. At a minimum, for each line data is collected from 34 fields of view from 6 wells across 3 independent experimental repeats. Data is plotted as a violin plot, with data plotted as per field or per well. When data is displayed as normalised to untreated control, each raw value has been divided by the average of the untreated control within each experimental repeat. An unpaired two-tailed student's t-test was used when comparing between two individual groups with gaussian distribution. When gaussian distribution was not achieved a Mann-Whitney test was used. Statistical analysis was conducted by Prism 8. A p value 0.05 or below was considered to be statistically significant (*p<0.05, **p<0.01, ***p<0.001).

### Example 2—TDP-43, SFPQ and FUS are Mislocalized to Neurites in VCP-Mutant Motor Neurons

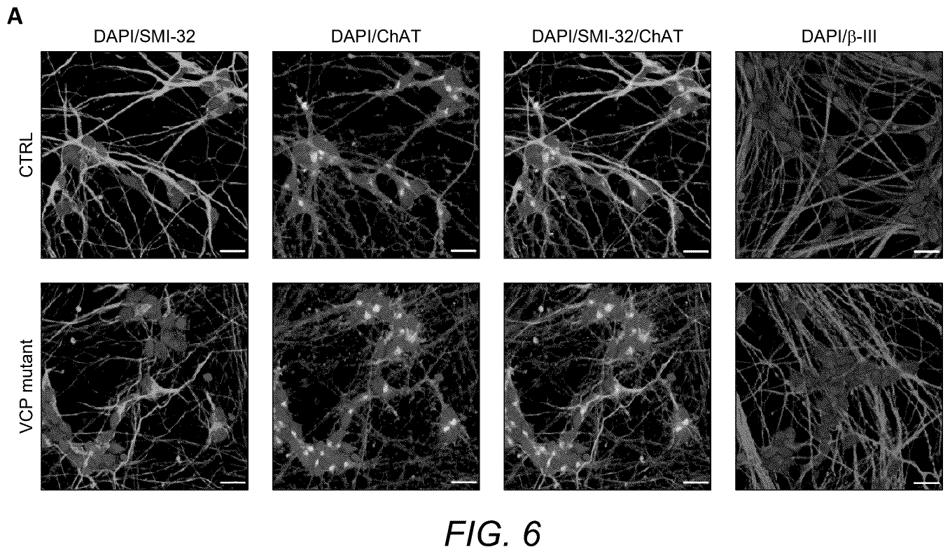

We have utilised our established and robust differentiation of human iPSCs into highly enriched and characterized spinal cord motor neurons (MNs), which were positive for choline acetyltransferase (ChAT), SMI-32 and βIII-tubulin (TUJ1) (FIG. 6).

Importantly, we have previously functionally validated our enriched MN cultures by i) demonstrating cytosolic calcium responses to physiological calcium stimuli (glutamate and KCl); ii) whole-cell patch clamping, iii) multi-electrode array (MEA) analysis [12] co-culture with iPSC-derived skeletal muscle with demonstration of neuromuscular junction formation [13]. Using this model, we have previously reported time-resolved pathogenic phenotypes of VCP-related ALS, including new hallmarks of ALS such as reduced SFPQ and FUS nuclear to cytoplasmic ratio in mutant neural precursors [2,3,12]. Additionally, we have confirmed subcellular TDP-43 and FUS mislocalization phenotypes in terminally differentiated motor neurons [12,29,30].

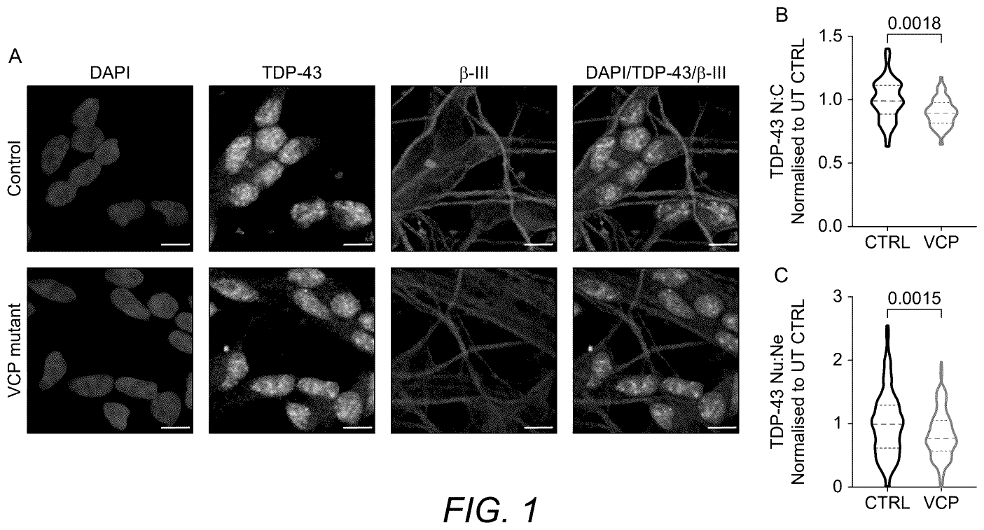

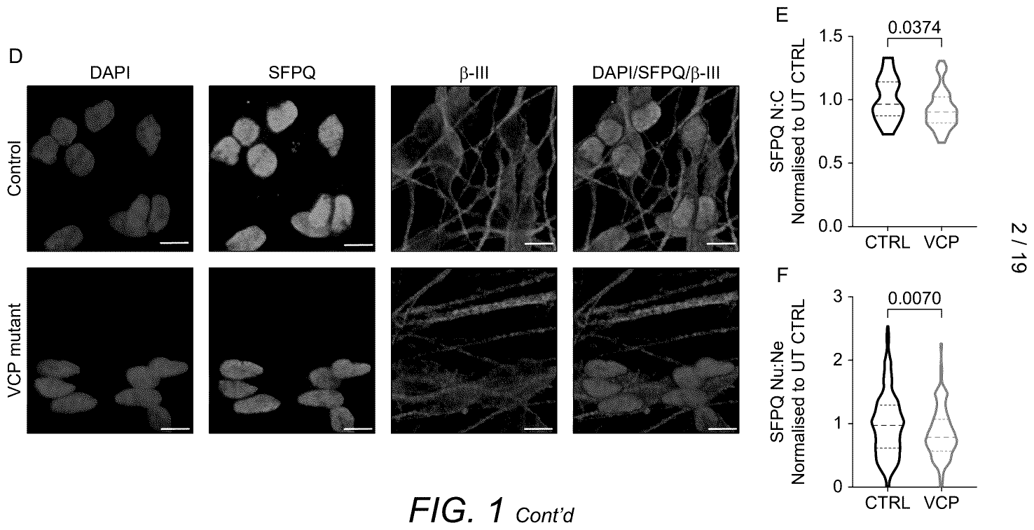

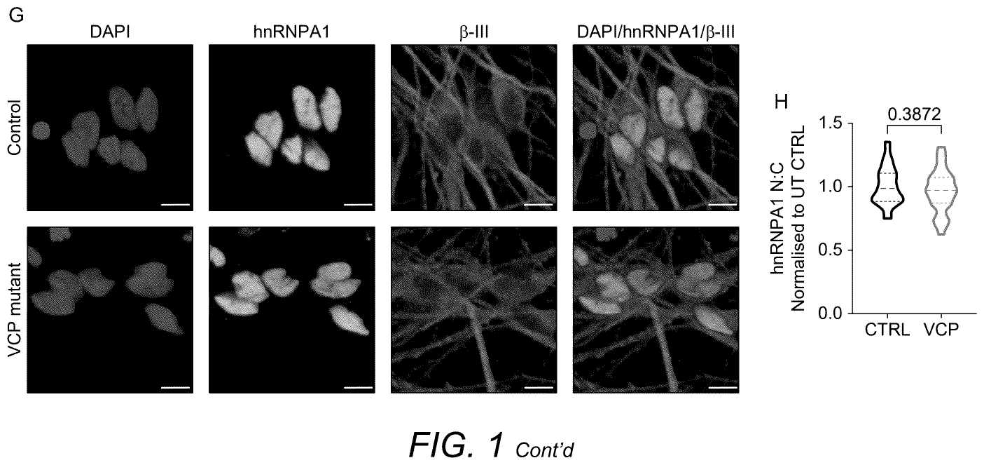

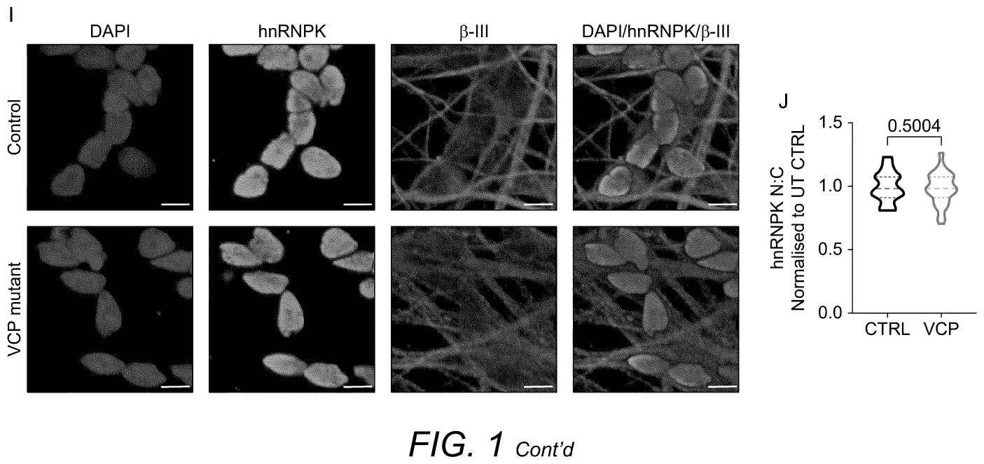

However, the majority of our prior studies and those of others have not systematically examined these aforementioned RBPs together, nor addressed the specific site of mislocalization of these RBPs with respect to their presence within neurites. Against this background, we utilised our VCP mutant iPSC-derived motor neurons to comprehensively investigate the subcellular localisation of 5 ALS-related RBPs. Single cell analysis of the nuclear-to-cytoplasmic ratio of >70,000 neurons revealed a decrease in TDP-43 and SFPQ in VCP-mutant human motor neurons (FIG. 1A, B, D, E), which builds on our recent report of reduced FUS nuclear-to-cytoplasmic ratio [29].

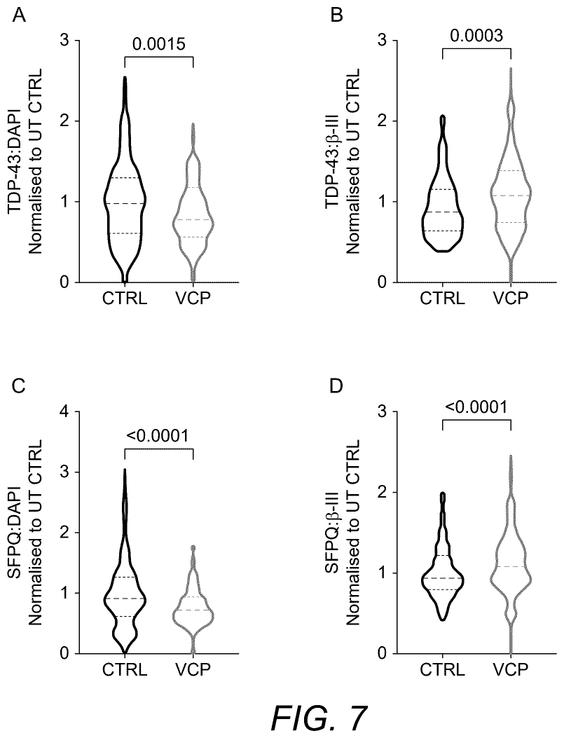

Upon further analysis, we detect that TDP-43 and SFPQ additionally have a reduced nuclear-to-neurite ratio and thus are also aberrantly localised within the neurites of VCP mutant motor neurons (FIG. 1C, F). The presence of compartment specific markers (nuclear:DAPI, neurites: βIII-tubulin) enabled us to then examine the nuclear and neurite compartments independently, revealing that the reduced nuclear-to-neurite ratios of these RBPs are driven by both their nuclear loss and neurite gain (FIG. 7A-D).

To exclude the possibility that that RBPs are generically mislocalized in iPSC models, we next examined the subcellular localisation of hnRNPA1 and hnRNPK, which have previously been implicated in ALS [31,32]. However, hnRNPA1 and hnRNPK exhibited no detectable change in their nuclear-to-cytoplasmic localization in VCP-mutant motor neurons consistent with selective mislocalization of TDP-43, FUS and SFPQ in iPSC-derived VCP mutant motor neurons (FIG. 1G-J).

### Example 3—Pharmacological Inhibition of the VCP D2 ATPase Domain does not Induce ALS Phenotypes in Healthy Human Motor Neurons

It has remained controversial in the field if VCP disease mutations exert dominant-active or dominant-negative effects. To gain mechanistic insight into the effect of the VCP mutations in human motor neurons, we utilized ML240, a potent and selective inhibitor of the D2 ATPase domain in the VCP protein [33].

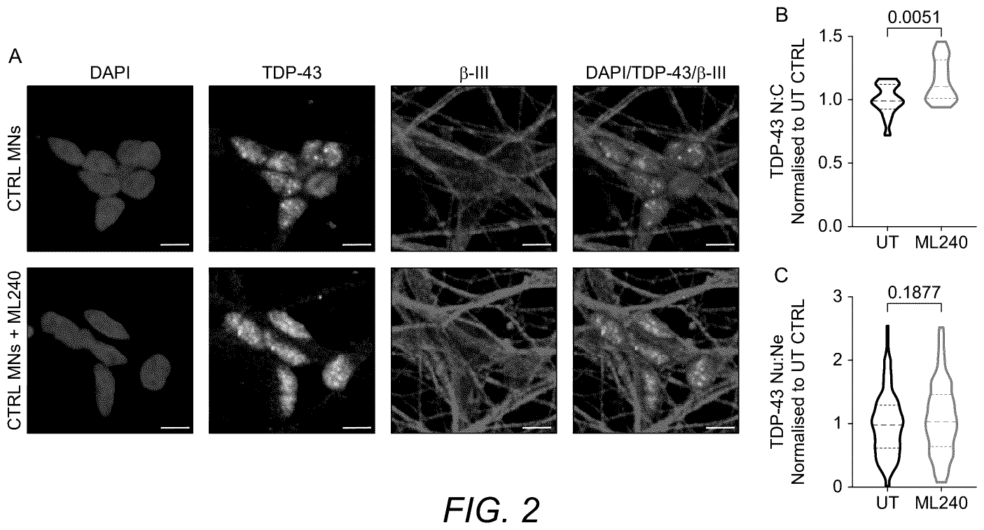

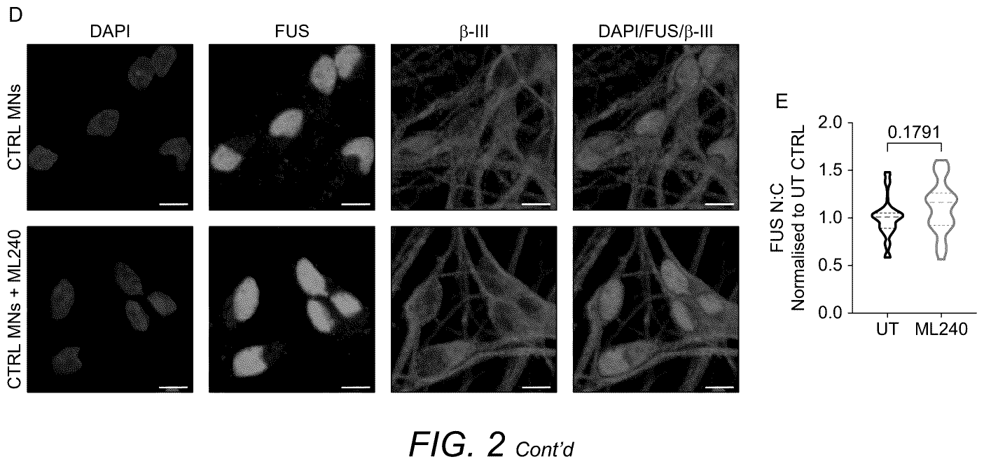

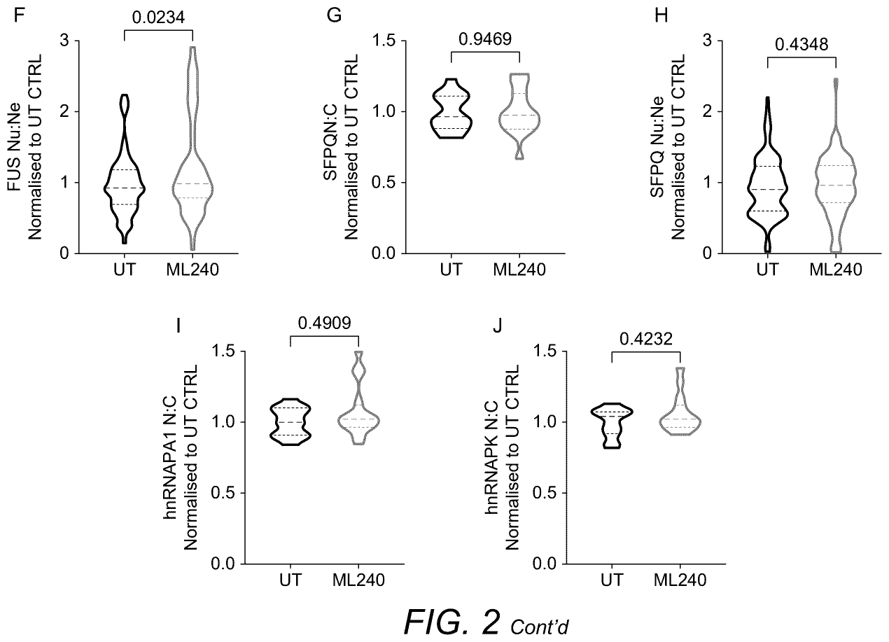

Control motor neurons were treated with 1 μM of ML240 for 2 hours prior to fixation and immunocytochemistry. Interestingly, inhibition of the D2 ATPase increased the nuclear-to-cytoplasmic ratio of TDP-43 (FIG. 2A, B). However, no such increase was observed in the nuclear-to-neurite ratio of TDP-43, possibly suggesting a proximal >distal change in protein distribution in this context (FIG. 2C).

Interestingly, we found that although FUS nuclear-to-cytoplasmic ratio did not change, a small but statistically significant increase in FUS nuclear-to-neurite ratio was observed (FIG. 2D-F). This suggests that the D2 ATPase domain may have RBP-specific roles in a cell compartment-specific manner. Analysis of the additional aforementioned RBPs; SFPQ, hnRNPA1 and hnRNPK revealed no changes in their nuclear-to-cytoplasmic ratios or nuclear-to-neurite ratio (SFPQ) upon VCP D2 ATPase inhibition (FIG. 2G-1). Together these data argue against a loss of function of the VCP D2 ATPase domain as a mechanism for the observed RBP mislocalization phenotypes.

### Example 4—Pharmacological Inhibition of the D2 ATPase Domain Reverses VCP Mutation-Related Mislocalization of TDP-43 and FUS in Human Motor Neurons

Noting the apparent effect of ML240 on TDP-43 in control motor neurons, we reasoned that its application to the VCP mutant motor neurons may ameliorate their RBP mislocalization phenotypes. Specifically, we hypothesised that the ALS VCP causing mutations (VCP R155C and VCP R191Q) result in a dominant-active effect of the D2 ATPase domain.

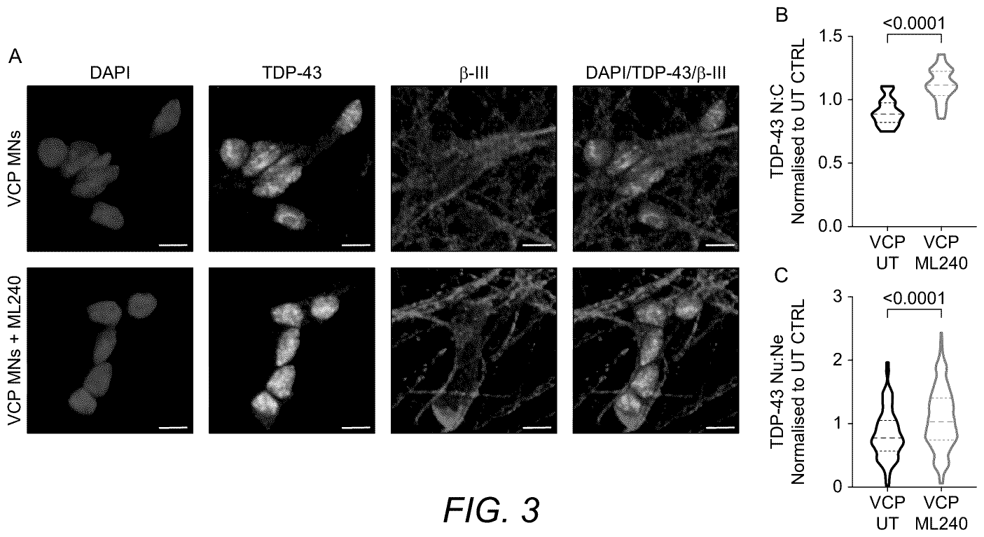

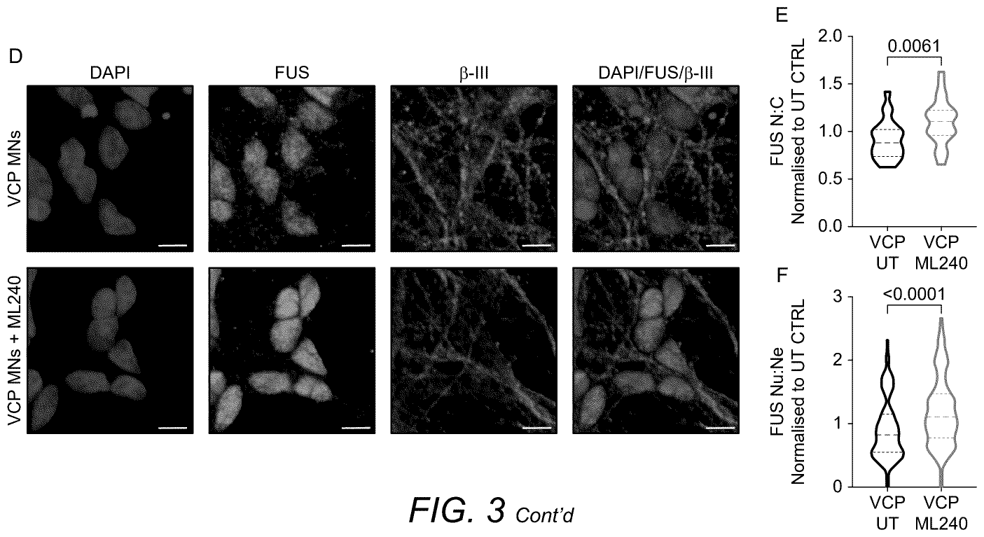

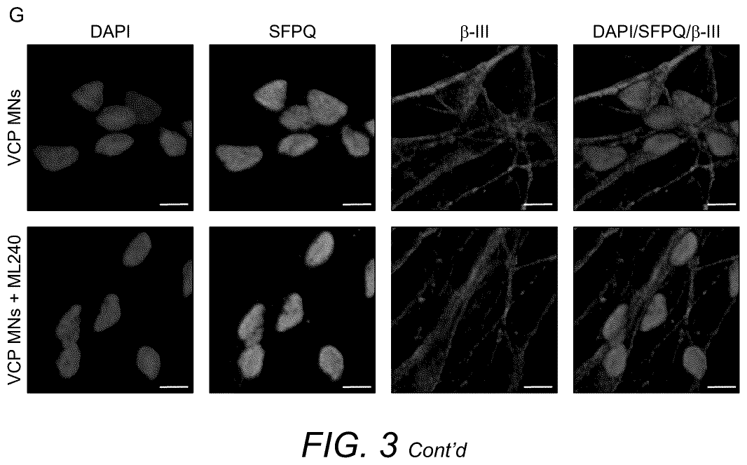

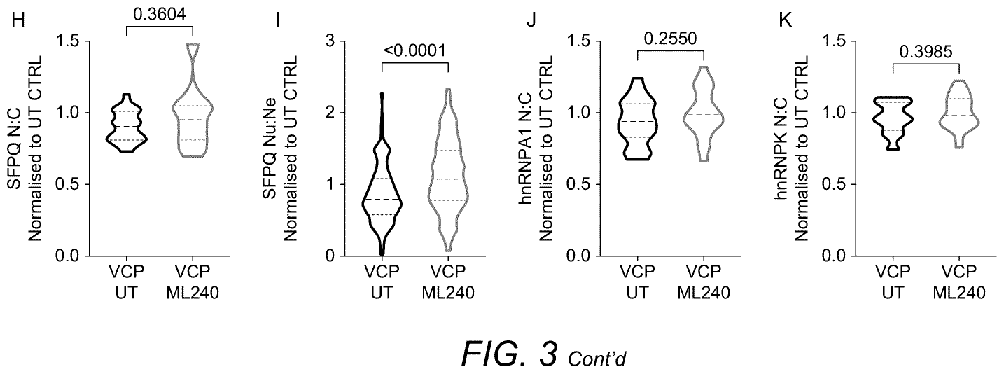

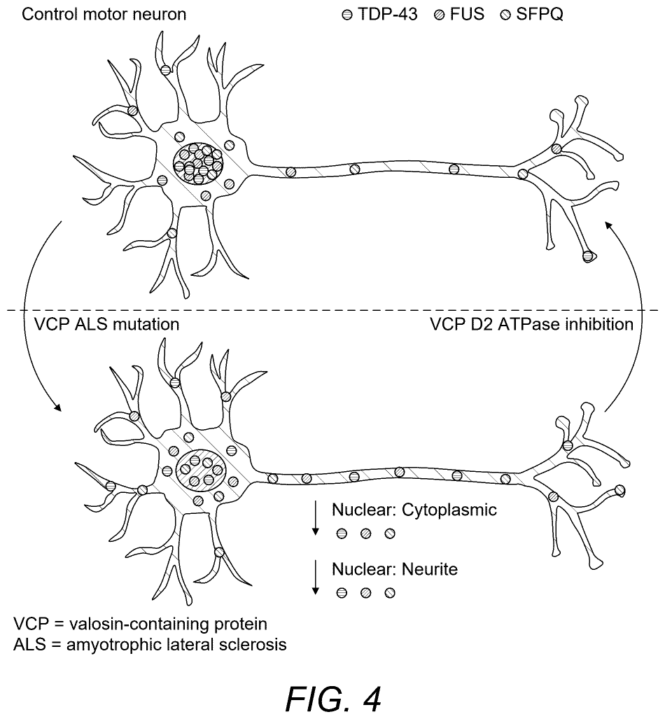

Application of ML240 in VCP mutant motor neurons indeed robustly reversed the mislocalization of both TDP-43 and FUS when examining both the nuclear-to-cytoplasmic mislocalization and nuclear-to-neurite mislocalization (FIG. 3A-F).

For SFPQ, ML240 treatment also significantly reversed the nuclear-to-neurite mislocalization. However, although ML240 treatment resulted in an increase in the nuclear-to-cytoplasmic ratio (Average; UT=0.90, ML240=0.97), this difference did not reach statistical significance for SFPQ (FIG. 3G-I).

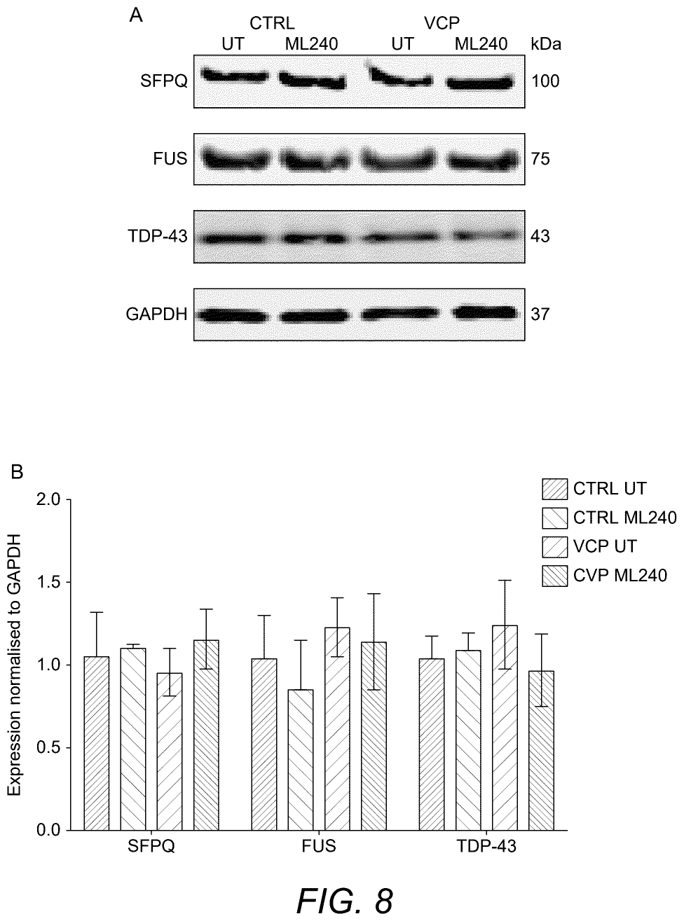

The data further demonstrates that this reversal is due to the relocalisation of TDP-43, FUS and SFPQ from the neurites and/or cytoplasm to the nucleus, as overall protein levels did not change upon ML240 treatment (FIG. 8A-B).

Notably, hnRNPA1 and hnRNPK exhibited no change in the nuclear-to-cytoplasmic ratio, with ML240 treatment only affecting the localisation of RBPs that were significantly mislocalized as a result of the VCP mutations (FIG. 3J, K).

In this study we have used our highly enriched and functionally validated iPSC-derived, patient specific motor neuron model [2,12,13] to systematically investigate the effects of ALS-causing VCP mutations on RBP nucleocytoplasmic localization and the ability of an inhibitor of the VCP D2 ATPase to suppress these VCP-related disease phenotypes. Importantly this model conveys mutations at pathophysiological levels and does not rely on artificial overexpression, thereby closely approximating physiology of ALS motor neurons.

The data presented here is the first to demonstrate that VCP mutations (R155C and R191Q) cause selective reduction in the nuclear-to-cytoplasmic ratios of TDP-43, FUS and SFPQ in terminally differentiated motor neurons, where a normal nucleocytoplasmic distribution of both hnRNPK and hnRNPA1 is observed. We further show that TDP-43, FUS and SFPQ are also mislocalized into the neurites of motor neurons (FIG. 1). Recent research has shown an emerging role for these RBPs in axonal mRNA translation and viability, suggesting that impaired axonal RNA processing could contribute to specific pathophysiology in motor neurons [34,35,36].

The main finding of our work, however, is that VCP-mutation related mislocalization of TDP-43, FUS and (in part) SPFQ is reversible through pharmacological inhibition of the D2 ATPase domain of VCP protein, using the potent and selective inhibitor ML240 [11,33,37]. This dominant-active VCP mutation mechanism has also been shown in relation to other phenotypes [38,39]. However, controversy exists in the field with some studies suggesting that VCP mutants function as dominant negatives [40,41,42].

These seemingly contrasting studies can be reconciled by raising the hypothesis that inhibiting ATP hydrolysis can reverse downstream effects associated with excess ATP hydrolysis whilst also increasing dominant negative effects on ATPase activation. Furthermore, as VCP has a wide range of intracellular functions, we can hypothesise that VCP mutations may result in both dominant active or negative effects, dependent on cofactor binding and subsequent downstream cellular pathways.

The underlying molecular mechanisms of how VCP interacts with TDP-43, FUS and SFPQ is unknown and further studies should investigate this. To date studies have shown a direct interaction between VCP and RBPs, including TDP-43 [43] and FUS [44], but with limited understanding of molecular consequences of these interactions.

A recent study in yeast showed a role for Cdc48/VCP in endocytosis-dependent turnover of TDP-43 and FUS [45]. Taken together with the present study, this raises the possibility that an increase in D2 ATPase activity may disrupt VCP-dependent endocytic mechanisms of cytoplasmic proteostasis. Understanding the precise consequences of VCP's interaction with RBPs will help decipher mechanisms underpinning their mislocalization in disease states.

Our findings that VCP disease mutants exhibit increased D2 ATPase activity have potential important therapeutic implications. Indeed, VCP inhibitors have been found to rescue multiple VCP disease phenotypes in drosophila models and patient fibroblasts [39].

Notably, rescue was multi-faceted including amelioration of mitochondrial phenotypes, p62 and ubiquitin pathology. In the context of our findings, this suggests that VCP D2 ATPase pharmacological inhibitors may be effective across the range of multi-system pathology caused by VCP mutations. This extends beyond ALS and IBMPFD into some cases of VCP-related Charcot-Marie-Tooth disease and hereditary spastic paraplegia [46,47]. However, as studies have shown that VCP inhibitors can perturb cellular homeostasis in a dose-dependent manner, a therapeutic balance must be investigated and optimised in future studies. However, with VCP inhibitors already in phase II clinical trials for cancer treatment, it is important to recognise the therapeutic potential for these devastating and hitherto incurable diseases.

### Example 5—Additional VCP Inhibitors Reverse Mislocalization of TDP-43 in VCP-Mutant and TARDBP-Mutant Human Motor Neurons

To gain further insight into the role the VCP inhibition in motor neurons, motor neurons were treated with additional VCP inhibitors, DBeQ and CB-5083. DBeQ is a reversible ATP competitive VCP inhibitor, that targets both the D1 and D2 ATPase domain of VCP. CB-5083 is a potent, reversible ATP competitive VCP inhibitor, that selectively targets the D2 ATPase domain.

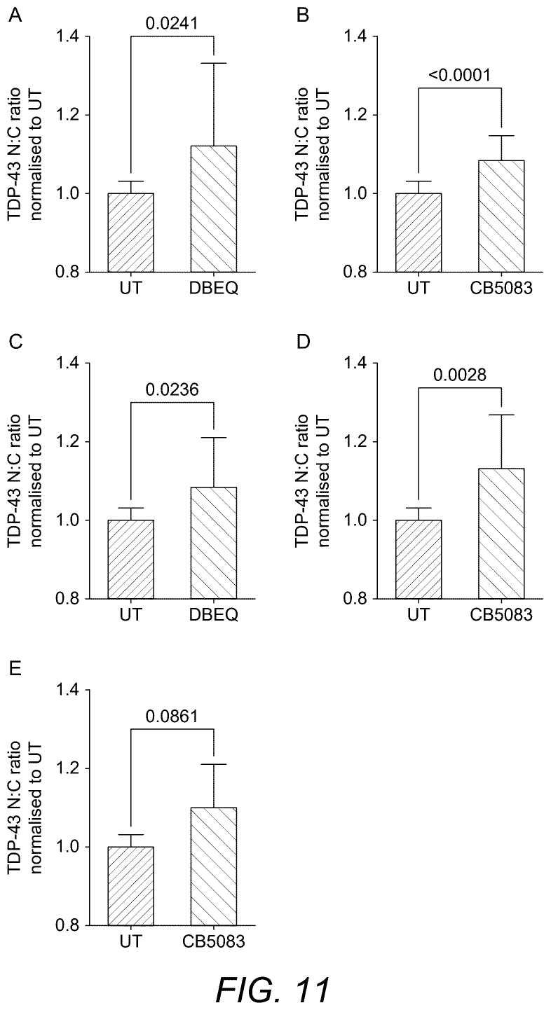

Control motor neurons were treated with 5 μM of DBeQ for 3 hours or 1 μM of CB-5083 for 3 hours prior to fixation and immunocytochemistry. Inhibition of VCP with each of DBeQ and CB-5083 increased the nuclear-to-cytoplasmic ratio of TDP-43 (FIG. 11A, B), supporting the findings observed upon ML240 treatment (see Example 3).

In VCP-mutant motor neurons, ML240 was shown to robustly reverse the mislocalization of TDP-43 (see Example 4). VCP-mutant motor neurons were treated with each of DBeQ and CB-5083. These additional VCP inhibitors were also able to reverse TDP-43 nuclear-to-cytoplasmic mislocalization (FIG. 11C, D).

To investigate if VCP inhibition reverses TDP-43 nuclear-to-cytoplasmic mislocalization in other ALS genetic backgrounds (i.e., non-VCP-mutants), TARDBP-mutant (G298S) motor neurons were treated with CB-5083. VCP inhibition with CB-5083 increased the nuclear-to-cytoplasmic ratio of TDP-43 in these cell lines (FIG. 11E).

These data demonstrate that VCP inhibitors other than ML240 are effective at reversing TDP-43 mislocalization in ALS mutant cell lines, both for VCP and non-VCP mutants.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

[1] Neumann M, Sampathu D M, Kwong L K, et al. Ubiquitinated TDP-43 in frontotemporal lobar degeneration and amyotrophic lateral sclerosis. Science. 2006; 314(5796):130-133.
[2] Luisier R, Tyzack G E, Hall C E, et al. Intron retention and nuclear loss of SFPQ are molecular hallmarks of ALS. Nat Commun. 2018; 9(1):2010.
[3] Tyzack G E, Luisier R, Taha D M, et al. Widespread FUS mislocalization is a molecular hallmark of amyotrophic lateral sclerosis. Brain. 2019; 142(9):2572-2580.
[4] Smethurst P, Newcombe J, Troakes C, et al. In vitro prion-like behaviour of TDP-43 in ALS. Neurobiol Dis. 2016; 96:236-247.
[5] Smethurst P, Risse E, Tyzack G E, et al. Distinct responses of neurons and astrocytes to TDP-43 proteinopathy in amyotrophic lateral sclerosis. Brain. 2020; 143(2):430-440.
[6] Tollervey J R, Curk T, Rogelj B, et al. Characterizing the RNA targets and position-dependent splicing regulation by TDP-43. Nat Neurosci. 2011; 14(4):452-458.
[7] Meyer H, Bug M, Bremer S. Emerging functions of the VCP/p97 AAA-ATPase in the ubiquitin system. Nat Cell Biol. 2012; 14(2):117-123.
[8] Johnson J O, Mandrioli J, Benatar M, et al. Exome sequencing reveals VCP mutations as a cause of familial ALS. Neuron. 2010; 68(5):857-864.
[9] Abramzon Y, Johnson J O, Scholz S W, et al. Valosin-containing protein (VCP) mutations in sporadic amyotrophic lateral sclerosis. Neurobiol Aging. 2012; 33(9):2231.e1-e2231.e6.
[10] Nalbandian A, Donkervoort S, Dec E, et al. The multiple faces of valosin-containing protein-associated diseases: inclusion body myopathy with Paget's disease of bone, frontotemporal dementia, and amyotrophic lateral sclerosis. J Mol Neurosci. 2011; 45(3):522-531.
[11] Niwa H, Ewens C A, Tsang C, Yeung H O, Zhang X, Freemont P S. The role of the N-domain in the ATPase activity of the mammalian AAA ATPase p97/VCP. J Biol Chem. 2012; 287(11):8561-8570.
[12] Hall C E, Yao Z, Choi M, et al. Progressive Motor Neuron Pathology and the Role of Astrocytes in a Human Stem Cell Model of VCP-Related ALS. Cell Rep. 2017; 19(9):1739-1749.
[13] Maffioletti S M, Sarcar S, Henderson A B H, et al. Three-Dimensional Human iPSC-Derived Artificial Skeletal Muscles Model Muscular Dystrophies and Enable Multilineage Tissue Engineering. Cell Rep. 2018; 23(3):899-908.
[14] Bursavich M G, Parker D P, Willardsen J A, et al (2010). 2-Anilino-4aryl-1,3-thiazole inhibitors of valosin-containing protein (VCP or p97). Bioorg Med Chem Lett, 20, 1677-9.
[15] Chou and Deshaies, Quantitative cell-based protein degradation assays to identify and classify drugs that target the ubiquitin-proteasome system, J Biol Chem. 2011 May 13; 286(19):16546-54. doi: 10.1074/jbc.M110.215319. Epub 2011 Feb. 22.
[16] Chou et al., Reversible inhibitor of p97, DBeQ, impairs both ubiquitin-dependent and autophagic protein clearance pathways, PNAS, Mar. 22, 2011 108 (12) 4834-4839
[17] Magnaghi et al., Abstract 2940: Identification of potent VCP/p97/CDC48 inhibitors with distinct biochemical mechanisms including a reversible, allosteric inhibitor that activates the unfolded protein response, induce autophagy and cancer cell death, Proceedings: AACR 103rd Annual Meeting 2012—Mar. 31-Apr. 4, 2012; Chicago, IL
[18] Wang, Q., Li, L, and Ye, Y. (2008). Inhibition of p97-dependent protein degradation by Eeyarestatin I. J Biol Chem 283, 7445-7454.
[19] Wang, Q., Shinkre, B. A., Lee, J. G., Weniger, M. A., Liu, Y., Chen, W., Wiestner, A., Trenkle, W. C., and Ye, Y. (2010). The ERAD inhibitor Eeyarestatin I is a bifunctional compound with a membrane-binding domain and a p97A CP inhibitory group. PLoS One 5, e15479.
[20] Sasazawa et al., Xanthohumol Impairs Autophagosome Maturation through Direct Inhibition of Valosin-Containing Protein, ACS Chem. Biol. 2012, 7, 5, 892-900.
[21] Madsen et al., New A TPase regulators—p97 goes to the PUB, Int J Biochem Cell Biol. 2009 December; 41(12):2380-8. doi: 10.1016/j.biocel.2009.05.017. Epub 2009 Jun. 2.
[22] Ritz, D., Vuk, M., Kirchner, P., Bug, M., Schutz, S., Hayer, A., Bremer, S., Lusk, C, Baloh, R. H., Lee, H et al. (2011). Endolysosomal sorting of ubiquitylated caveolin-1 is regulated by VCP and UBXD1 and impaired by VCP disease mutations. Nat Cell Biol 13, 1116-1123.
[23] Yamanaka et al., Recent advances in p97/VCP/Cdc48 cellular functions, (2012) Biochim Biophys Acta. 2012 January; 1823(1):130-7.
[24] Ingre et al., Risk factors for amyotrophic lateral sclerosis, Clin Epidemiol. 2015; 7: 181-193. Published online 2015 Feb. 12. doi: 10.2147/CLEP.S37505
[25] Byrne S, Bede P, Elamin M, et al. Proposed criteria for familial amyotrophic lateral sclerosis. Amyotroph Lateral Scler. 2011; 12(3):157-159.
[26] Okita, K., Matsumura, Y., Sato, Y. et al. A more efficient method to generate integration-free human iPS cells. Nat Methods 8, 409-412 (2011). https://doi.org/10.1038/nmeth.1591
[27] Berg S, Kutra D, Kroeger T, et al. ilastik: interactive machine learning for (bio)image analysis. Nat Methods. 2019; 16(12):1226-1232.
[28] Carpenter A E, Jones T R, Lamprecht M R, et al. CellProfiler: image analysis software for identifying and quantifying cell phenotypes. Genome Biol. 2006; 7(10):R100.
[29] Harley J, Hagemann C, Serio A, Patani R. FUS is lost from nuclei and gained in neurites of motor neurons in a human stem cell model of VCP-related ALS. Brain. 2020; 143(12):e103.
[30] Harley J, Patani R. Stress-Specific Spatiotemporal Responses of RNA-Binding Proteins in Human Stem-Cell-Derived Motor Neurons. Int J Mol Sci. 2020; 21(21). doi:10.3390/ijms21218346
[31] Purice M D, Taylor J P. Linking hnRNP Function to ALS and FTD Pathology. Front Neurosci. 2018; 12:326.
[32] Moujalled D, Grubman A, Acevedo K, et al. TDP-43 mutations causing amyotrophic lateral sclerosis are associated with altered expression of RNA-binding protein hnRNP K and affect the Nrf2 antioxidant pathway. Hum Mol Genet. 2017; 26(9):1732-1746.
[33] Chou T-F, Li K, Frankowski K J, Schoenen F J, Deshaies R J. Structure-activity relationship study reveals ML240 and ML241 as potent and selective inhibitors of p97 ATPase. ChemMedChem. 2013; 8(2):297-312.
[34] Briese M, Saal-Bauernschubert L, Luningschror P, et al. Loss of Tdp-43 disrupts the axonal transcriptome of motoneurons accompanied by impaired axonal translation and mitochondria function. Acta Neuropathol Commun. 2020; 8(1):116.
[35] Cosker K E, Fenstermacher S J, Pazyra-Murphy M F, Elliott H L, Segal R A. The RNA-binding protein SFPQ orchestrates an RNA regulon to promote axon viability. Nature Neuroscience. 2016; 19(5):690-696. doi:10.1038/nn.4280.
[36] Lopez-Erauskin J, Tadokoro T, Baughn M W, et al. ALS/FTD-Linked Mutation in FUS Suppresses Intra-axonal Protein Synthesis and Drives Disease Without Nuclear Loss-of-Function of FUS. Neuron. 2020; 106(2):354.
[37] Manno A, Noguchi M, Fukushi J, Motohashi Y, Kakizuka A. Enhanced ATPase activities as a primary defect of mutant valosin-containing proteins that cause inclusion body myopathy associated with Paget disease of bone and frontotemporal dementia. Genes Cells. 2010; 15(8):911-922.
[38] Chang Y-C, Hung W-T, Chang Y-C, et al. Pathogenic VCP/TER94 Alleles Are Dominant Actives and Contribute to Neurodegeneration by Altering Cellular ATP Level in a Drosophila IBMPFD Model. PLoS Genetics. 2011; 7(2):e1001288. doi:10.1371/journal.pgen.1001288
[39] Zhang T, Mishra P, Hay B A, Chan D, Guo M. Valosin-containing protein (VCP/p97) inhibitors relieve Mitofusin-dependent mitochondrial defects due to VCP disease mutants. Elife. 2017; 6. doi:10.7554/eLife.17834
[40] Bartolome F, Wu H-C, Burchell V S, et al. Pathogenic VCP Mutations Induce Mitochondrial Uncoupling and Reduced ATP Levels. Neuron. 2013; 78(1):57-64. doi:10.1016/j.neuron.2013.02.028.
[41] Ritz D, Vuk M, Kirchner P, et al. Endolysosomal sorting of ubiquitylated caveolin-1 is regulated by VCP and UBXD1 and impaired by VCP disease mutations. Nat Cell Biol. 2011; 13(9):1116-1123.
[42] Rycenga H B, Wolfe K B, Yeh E S, Long D T. Uncoupling of p97 ATPase activity has a dominant negative effect on protein extraction. Sci Rep. 2019; 9(1):1-11.
[43] Gitcho M A, Strider J, Carter D, et al. VCP mutations causing frontotemporal lobar degeneration disrupt localization of TDP-43 and induce cell death. J Biol Chem. 2009; 284(18):12384-12398.
[44] Wang T, Jiang X, Chen G, Xu J. Interaction of amyotrophic lateral sclerosis/frontotemporal lobar degeneration-associated fused-in-sarcoma with proteins involved in metabolic and protein degradation pathways. Neurobiol Aging. 2015; 36(1):527-535.
[45] Liu G, Byrd A, Warner A N, et al. Cdc48/VCP and Endocytosis Regulate TDP-43 and FUS Toxicity and Turnover. Mol Cell Biol. 2020; 40(4). doi:10.1128/MCB.00256-19.
[46] Gonzalez M A, Feely S M, Speziani F, et al. A novel mutation in VCP causes Charcot-Marie-Tooth Type 2 disease. Brain. 2014; 137(Pt 11):2897-2902.
[47] de Bot S T, Schelhaas H J, Kamsteeg E-J, van de Warrenburg B P C. Hereditary spastic paraplegia caused by a mutation in the VCP gene. Brain. 2012; 135(Pt 12):e223; author reply e224.

1. A method of treating or preventing amyotrophic lateral sclerosis (ALS) comprising administering VCP (Valosin-containing protein) inhibitor to a subject in need thereof, wherein the subject has no known disease-causing mutations in a VCP gene, SOD1 gene, ALS2 gene, VAPB gene, SETX gene, TDP-43 gene, FUS/TLS gene, and/or OPTN gene.

2. The method according to claim 1, wherein the subject has not been identified as having a disease-causing mutation in a VCP gene.

3. The method according to claim 1, wherein the subject has been identified as not having a disease-causing mutation in a VCP gene.

4. The method according to claim 1, wherein the ALS is non VCP-associated ALS.

5. The method according to claim 1, wherein the subject has been identified as not having a disease-causing genetic mutation in a VCP gene
i) at any of positions R155 and R191;
ii) selected from the list consisting of: R155C and R191Q;
iii) at any of positions R95, I114, I151, R155, G156, M158, R159, R191, N387, N401, R487, D592, R662 and N750; or
iv) selected from the list consisting of: R95C, R95G, I114V, I151V, R155H, R155C, G156C, M158V, R159G, R159C, R159H, R191G, R191Q, N387T, N401S, R487H, D592N, R662C and N750S.

6-9. (canceled)

10. The method according to claim 1, wherein the amyotrophic lateral sclerosis is associated with reduction in the nuclear-to-cytoplasmic ratios of one or more of TDP-43, FUS and/or SFPQ, optionally wherein the VCP inhibitor ameliorates one or more symptoms associated with reduction in the nuclear-to-cytoplasmic ratios of one or more of TDP-43, FUS and/or SFPQ; optionally wherein the amyotrophic lateral sclerosis is associated with reduction in the nuclear-to-cytoplasmic ratio of:
i) TDP 43, optionally wherein the VCP inhibitor ameliorates one or more symptoms associated with reduction in the nuclear-to-cytoplasmic ratios of TDP-43;
ii) FUS, optionally wherein the VCP inhibitor ameliorates one or more symptoms associated with reduction in the nuclear-to-cytoplasmic ratios of FUS; or
iii) SFPQ, optionally wherein the VCP inhibitor ameliorates one or more symptoms associated with reduction in the nuclear-to-cytoplasmic ratios of SFPQ.

11-13. (canceled)

14. The method according to claim 1, wherein treating or preventing ALS comprises partial or complete alleviation, amelioration, relief, inhibition, delaying onset, reducing severity and/or incidence of neurological impairment in a patient suffering from or susceptible to ALS; optionally wherein neurological impairment comprises symptoms associated with impairment of the central nervous system such as one or more of developmental delay, progressive cognitive impairment, hearing loss, impaired speech development, deficits in motor skills, hyperactivity, aggressiveness and/or sleep disturbances; optionally wherein treating or preventing ALS with a VCP inhibitor results in an improvement or amelioration of one or more neurological impairment symptoms by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%, as compared to the neurological impairment symptoms in the absence of a VCP inhibitor.

15-16. (canceled)

17. The method according to claim 1, wherein the VCP inhibitor inhibits the D2 ATPase domain of VCP.

18. The method according to claim 1, wherein the VCP inhibitor is selected from the group consisting of: ML240 (2-(2-Amino-1H-benzimidazole-1-yl)-8-methoxy-N-(phenylmethyl)-4-quinazolinamine), ML241, 2-anilino-4-aryl-1,3-thiazoles, 3,4-methylenedioxy-6-nitrostyrene, DBeQ (N2,N4-dibenzylquinazo-line-2,4-diamine), CB-5083 (1-[7,8-dihydro-4-[(phenylmethyl)amino]-5H-pyrano[4,3-d]pyrimidin-2-yl]-2-methyl-1H-indole-4-carboxamide), CB-5339 (1-[4-(Benzylamino)-5,6,7,8-tetrahydropyrido[2,3-d]pyrimidin-2-yl]-2-methylindole-4-carboxamide), UPCDC-30245 (1-(3-(5-Fluoro-1H-indol-2-yl)phenyl)-N-(2-(4-isopropylpiperazin-1-yl)ethyl)piperidin-4-amine), NMS-873, NMS-859, Eeyarestatin I and Xanthohumol; optionally
i) wherein the VCP inhibitor is selected from the group consisting of: ML240 (2-(2-Amino-1H-benzimidazole-1-yl)-8-methoxy-N-(phenylmethyl)-4-quinazolinamine), ML241, 2-anilino-4-aryl-1,3-thiazoles, 3,4-methylenedioxy-6-nitrostyrene, DBeQ (N2,N4-dibenzylquinazo-line-2,4-diamine), NMS-873, NMS-859, Eeyarestatin I and Xanthohumol; optionally wherein the VCP inhibitor is ML240 (2-(2-Amino-1H-benzimidazole-1-yl)-8-methoxy-N-(phenylmethyl)-4-quinazolinamine); or
ii) wherein the VCP inhibitor is CB-5083 (1-[7,8-dihydro-4-[(phenylmethyl)amino]-5H-pyrano[4,3-d]pyrimidin-2-yl]-2-methyl-1H-indole-4-carboxamide) or CB-5339 (1-[4-(Benzylamino)-5,6,7,8-tetrahydropyrido[2,3-d]pyrimidin-2-yl]-2-methylindole-4-carboxamide).

19-26. (canceled)

27. A pharmaceutical composition comprising a VCP inhibitor for use in a method of treating or preventing amyotrophic lateral sclerosis (ALS) in a subject, wherein the subject has no known disease-causing mutations in a VCP gene, SOD1 gene, ALS2 gene, VAPB gene, SETX gene, TDP-43 gene, FUS/TLS gene, and/or OPTN gene optionally wherein the pharmaceutical composition comprises one or more excipients.

28-31. (canceled)

32. A kit for diagnosing a subject as having or being suspected of having non-VCP-associated ALS comprising a means of determining whether the subject has a disease-causing mutation in a VCP gene; optionally further comprising one or more containers containing one or more VCP inhibitors and, optionally informational material; optionally wherein the informational material comprises directions for use of the kit in the diagnosis and treatment of non-VCP-associated ALS.

33-34. (canceled)

35. The pharmaceutical composition according to claim 27, wherein the VCP inhibitor:
a) inhibits the D2 ATPase domain of VCP; or
b) is selected from the group consisting of: ML240 (2-(2-Amino-1H-benzimidazole-1-yl)-8-methoxy-N-(phenylmethyl)-4-quinazolinamine), ML241, 2-anilino-4-aryl-1,3-thiazoles, 3,4-methylenedioxy-6-nitrostyrene, DBeQ (N2,N4-dibenzylquinazo-line-2,4-diamine), CB-5083 (1-[7,8-dihydro-4-[(phenylmethyl)amino]-5H-pyrano[4,3-d]pyrimidin-2-yl]-2-methyl-1H-indole-4-carboxamide), CB-5339 (1-[4-(Benzylamino)-5,6,7,8-tetrahydropyrido[2,3-d]pyrimidin-2-yl]-2-methylindole-4-carboxamide), UPCDC-30245 (1-(3-(5-Fluoro-1H-indol-2-yl)phenyl)-N-(2-(4-isopropylpiperazin-1-yl)ethyl)piperidin-4-amine), NMS-873, NMS-859, Eeyarestatin I and Xanthohumol; optionally
i) wherein the VCP inhibitor is selected from the group consisting of: ML240 (2-(2-Amino-1H-benzimidazole-1-yl)-8-methoxy-N-(phenylmethyl)-4-quinazolinamine), ML241, 2-anilino-4-aryl-1,3-thiazoles, 3,4-methylenedioxy-6-nitrostyrene, DBeQ (N2,N4-dibenzylquinazo-line-2,4-diamine), NMS-873, NMS-859, Eeyarestatin I and Xanthohumol; optionally wherein the VCP inhibitor is ML240 (2-(2-Amino-1H-benzimidazole-1-yl)-8-methoxy-N-(phenylmethyl)-4-quinazolinamine); or
ii) wherein the VCP inhibitor is CB-5083 (1-[7,8-dihydro-4-[(phenylmethyl)amino]-5H-pyrano[4,3-d]pyrimidin-2-yl]-2-methyl-1H-indole-4-carboxamide) or CB-5339 (1-[4-(Benzylamino)-5,6,7,8-tetrahydropyrido[2,3-d]pyrimidin-2-yl]-2-methylindole-4-carboxamide).

36. The kit of claim 32, wherein the VCP inhibitor:
a) inhibits the D2 ATPase domain of VCP; or
b) is selected from the group consisting of: ML240 (2-(2-Amino-1H-benzimidazole-1-yl)-8-methoxy-N-(phenylmethyl)-4-quinazolinamine), ML241, 2-anilino-4-aryl-1,3-thiazoles, 3,4-methylenedioxy-6-nitrostyrene, DBeQ (N2,N4-dibenzylquinazo-line-2,4-diamine), CB-5083 (1-[7,8-dihydro-4-[(phenylmethyl)amino]-5H-pyrano[4,3-d]pyrimidin-2-yl]-2-methyl-1H-indole-4-carboxamide), CB-5339 (1-[4-(Benzylamino)-5,6,7,8-tetrahydropyrido[2,3-d]pyrimidin-2-yl]-2-methylindole-4-carboxamide), UPCDC-30245 (1-(3-(5-Fluoro-1H-indol-2-yl)phenyl)-N-(2-(4-isopropylpiperazin-1-yl)ethyl)piperidin-4-amine), NMS-873, NMS-859, Eeyarestatin I and Xanthohumol; optionally
i) wherein the VCP inhibitor is selected from the group consisting of: ML240 (2-(2-Amino-1H-benzimidazole-1-yl)-8-methoxy-N-(phenylmethyl)-4-quinazolinamine), ML241, 2-anilino-4-aryl-1,3-thiazoles, 3,4-methylenedioxy-6-nitrostyrene, DBeQ (N2,N4-dibenzylquinazo-line-2,4-diamine), NMS-873, NMS-859, Eeyarestatin I and Xanthohumol; optionally wherein the VCP inhibitor is ML240 (2-(2-Amino-1H-benzimidazole-1-yl)-8-methoxy-N-(phenylmethyl)-4-quinazolinamine); or
ii) wherein the VCP inhibitor is CB-5083 (1-[7,8-dihydro-4-[(phenylmethyl)amino]-5H-pyrano[4,3-d]pyrimidin-2-yl]-2-methyl-1H-indole-4-carboxamide) or CB-5339 (1-[4-(Benzylamino)-5,6,7,8-tetrahydropyrido[2,3-d]pyrimidin-2-yl]-2-methylindole-4-carboxamide).


SEQUENCE LISTING (1 sequence)

SEQ ID NO: 1 - protein, 806 aa, Homo sapiens
MASGADSKGDDLSTAILKQKNRPNRLIVDEAINEDNSVVSLSQPKMDELQLFRGDTVLLKGKKRREAVCIVLSDDTCSDEKIRMNRVVRNNLRVRLGDVISIQPCPDVKYGKRIHVLPIDDTVEGITGNLFEVYLKPYFLEAYRPIRKGDIFLVRGGMRAVEFKVVETDPSPYCIVAPDTVIHCEGEPIKREDEEESLNEVGYDDIGGCRKQLAQIKEMVELPLRHPALFKAIGVKPPRGILLYGPPGTGKTLIARAVANETGAFFFLINGPEIMSKLAGESESNLRKAFEEAEKNAPAIIFIDELDAIAPKREKTHGEVERRIVSQLLTLMDGLKQRAHVIVMAATNRPNSIDPALRRFGRFDREVDIGIPDATGRLEILQIHTKNMKLADDVDLEQVANETHGHVGADLAALCSEAALQAIRKKMDLIDLEDETIDAEVMNSLAVTMDDFRWALSQSNPSALRETVVEVPQVTWEDIGGLEDVKRELQELVQYPVEHPDKFLKFGMTPSKGVLFYGPPGCGKTLLAKAIANECQANFISIKGPELLTMWFGESEANVREIFDKARQAAPCVLFFDELDSIAKARGGNIGDGGGAADRVINQILTEMDGMSTKKNVFIIGATNRPDIIDPAILRPGRLDQLIYIPLPDEKSRVAILKANLRKSPVAKDVDLEFLAKMTNGFSGADLTEICQRACKLAIRESIESEIRRERERQTNPSAMEVEEDDPVPEIRRDHFEEAMRFARRSVSDNDIRKYEMFAQTLQQSRGFGSFRFPSGNQGGAGPSQGSGGGTGGSVYTEDNDDDLYG